(19) United States
(12) Patent Application Publication
(10) Pub. No.: US 2025/0257044 A1
(43) Pub. Date: Aug. 14, 2025

(54) METHOD FOR PREPARING CARONIC ANHYDRIDE

(71) Applicant: ABA CHEMICALS NANTONG LIMITED, Nantong, Jiangsu Province (CN)

(72) Inventors: Zhigang LIN, Nantong, Jiangsu Province (CN); Limin QUE, Nantong, Jiangsu Province (CN); Guangsheng SHAN, Nantong, Jiangsu Province (CN); Panpan GAO, Nantong, Jiangsu Province (CN); Chengxiang CHEN, Nantong, Jiangsu Province (CN)

(73) Assignee: ABA CHEMICALS NANTONG LIMITED, Nantong, Jiangsu Province (CN)

(21) Appl. No.: 18/867,029

(22) PCT Filed: Nov. 18, 2022
(86) PCT No.: PCT/CN2022/132714

§ 371 Date: Nov. 19, 2024

(30) Foreign Application Priority Data
May 23, 2022 (CN) 202210566949.2

(51) Int. Cl. C07D 307/93

(52) U.S. Cl. CPC C07D 307/93

(57) ABSTRACT

The present disclosure relates to a method for preparing caronic anhydride. The method comprises cyclizing caronic acid to obtain crude caronic anhydride; and using isopropyl ether to recrystallize the crude caronic anhydride to obtain caronic anhydride.

### TECHNICAL FIELD

The present disclosure relates to a method for preparing caronic anhydride.

### BACKGROUND ART

Caronic anhydride is an important pharmaceutical intermediate, which is mainly used to produce hepatitis C protease inhibitor boceprevir and oral COVID-19 drug Paxlovid and has a great market potential. In the prior art, caronic anhydride at high purity is generally prepared by first preparing caronic acid, then cyclizing the caronic acid at a high temperature under the action of acetic anhydride to obtain crude caronic anhydride, and finally recrystallizing the crude product.

There are several methods for synthesizing caronic acid in the prior art. For example, conventionally, caronic acid is synthesized from chrysanthemum monocarboxylate through oxidation, saponification and acidification, but this method has the disadvantages of high raw material price, dangerous operation (potassium permanganate is used), environmental pollution and high production cost. In addition, patent CN104163759B discloses a new method for preparing caronic acid, by which isopentenol containing protected hydroxyl is used as a starting material, a key intermediate with a three-membered ring is generated through the addition of double bonds, then ethyl ester and protecting group are hydrolyzed and then the oxidation conditions are controlled. The new method can produce caronic acid in a more economical, safer, more environment-friendly and cheaper manner, but it still has the disadvantages of undesirable purity and yield of the caronic acid prepared.

For the cyclization and recrystallization of caronic acid, the prior art still has some disadvantages, such as low yields of cyclization and recrystallization, low purity of the product and high toxicity of the recrystallization solvent, which need to be improved urgently.

Therefore, it is very necessary to provide a new method for preparing caronic anhydride, which can obtain high-quality caronic anhydride with a higher yield, a higher purity, a lower cost and a more environmentally friendly method.

### SUMMARY OF THE INVENTION

In order to solve the foregoing problem, the present disclosure proposes a new method for preparing caronic anhydride. By improving the conditions of cyclization and recrystallization of caronic acid, the method effectively improves the yield and purity of the product after cyclization and recrystallization and reduces reaction toxicity. Further, by improving one or more of the reaction parameters (such as oxidation conditions, reaction temperature, pH and crystallization conditions) of the method for preparing caronic acid disclosed in patent CN104163759B, this method effectively improves the purity and yield of product caronic acid.

According to the first aspect of the present disclosure, a method for preparing caronic anhydride is provided, which comprises the following steps:
s1) cyclizing caronic acid to obtain crude caronic anhydride; and
s2) using isopropyl ether to recrystallize the crude caronic anhydride to obtain caronic anhydride.

In some embodiments, the step s1) comprises:
s11) causing the reaction mixture prepared by mixing a starting material to react, wherein the starting material contains:
caronic acid
a catalyst; and
a solvent; and
s12) distilling, preferably distilling under atmospheric pressure, the product obtained in the above step s11) to separate and obtain crude caronic anhydride.

In some embodiments, in the first aspect:
In the step s11), the catalyst is one or more of: sodium acetate, sodium hydroxide, sodium bicarbonate and sodium carbonate, preferably sodium acetate; and/or
In the step s11), the solvent is acetic anhydride; and/or
In the step s11), the weight ratio of the caronic acid, the catalyst and the solvent is 1:(0.01-0.05): (1-3), preferably 1:0.03:2; and/or
The step s11) is carried out at 130-140° C., preferably 135-138° C. for 1-5 h, preferably 2-4 h, more preferably 3 h; and/or

The step s12) comprises:
s121) distilling under atmospheric pressure at 140-145° C. the product obtained in the step s11) for 3-8 h to obtain a post-distillation product;
s122) heating and treating the post-distillation product, preferably in a high-temperature reactor, to collect a fraction of 165-200° C.;
s123) maintaining the temperature of the fraction obtained in the step s122) at 190-200° C. for 3-6 h to obtain crude caronic anhydride.

In some embodiments, the step s2) comprises:
s21) dissolving the crude caronic anhydride in isopropyl ether at a high temperature, preferably 50-70° C., more preferably 55-60° C. to obtain a clear solution; and
s22) cooling the clear solution obtained in the above step s21) to a low temperature, preferably to 3-10° C., more preferably to 5-8° C. to precipitate a solid, i.e., caronic anhydride.

In some embodiments, the caronic acid in the step s1) is prepared by the method comprising the following steps:
a) causing a compound of Formula (I) to react with a compound of Formula (II) to obtain a compound of Formula (III),

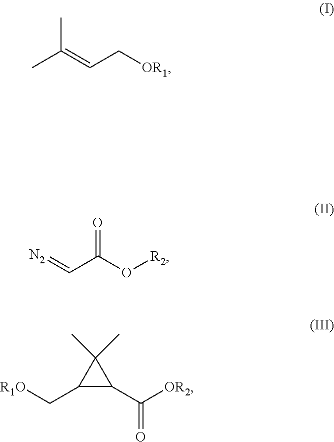

where the R₁ is a protecting group, preferably, the R₁ is selected from an ester protecting group, an alkyl ether protecting group and a silyl ether protecting group, more preferably, the ester protecting group is selected from acetyl, benzoyl and substituted benzoyl, the alkyl ether protecting group is selected from benzyl, triphenyl-methyl and tetrahydropyranyl, and the silyl ether protecting group is selected from trimethylsilyl and dimethyltert-butylsilyl, and most preferably, the protecting group is acetyl;

The R₂ is selected from: alkyl, cycloalkyl, aryl, alkylaryl, heterocyclyl and heteroaryl, preferably, the R₂ is selected from substituted or unsubstituted alkyl, more preferably, the R₂ is selected from unsubstituted alkyl, most preferably, the R₂ is ethyl;
b) hydrolyzing the compound of Formula (III) to obtain a compound of Formula (IV),

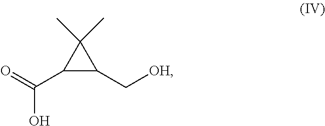

c) oxidizing the compound of Formula (IV) and obtaining caronic acid.

In some embodiments, the step c) comprises using a TEMPO oxidation system to oxidize the compound of Formula (IV) and obtain caronic acid,
The TEMPO oxidation system comprises:
TEMPO;
bicarbonate;
bromide; and
hypochlorite.

In some embodiments, wherein:
The bicarbonate is sodium bicarbonate; and/or
The bromide is potassium bromide; and/or
The hypochlorite is sodium hypochlorite; and/or
The weight ratio of the compound of Formula (IV), the TEMPO, the bicarbonate, the potassium bromide and the hypochlorite is 1:(0.01-0.05): (0.3-1): (0.01-0.1): (0.9-1.4), preferably 1:(0.02-0.04): (0.5-0.8): (0.02-0.08): (1-1.3), more preferably 1:(0.02-0.04): (0.5-0.7): (0.04-0.06): (1-1.2), most preferably 1:0.03:0.62:0.052:1.08.

In some embodiments, the step a) comprises:
a1) causing the reaction mixture prepared by mixing a starting material to react, wherein the starting material contains:
a compound of Formula (I);
a compound of Formula (II);
a catalyst; and
a solvent; and
a2) separating from the reaction product obtained in the step a1) to obtain the compound of Formula (III).

Preferably, wherein:
In the step a1), the weight ratio of the compound of Formula (I), the compound of Formula (II), the catalyst and the solvent is 1: (0.5-1.0): (0.005-0.015): (1.5-2.5), preferably 1: (0.6-0.8): (0.006-0.01): (1.6-2), more preferably 1: (0.6-0.8): (0.007-0.09): (1.7-1.9), most preferably 1:0.74:0.008:1.8; and/or
The step a1) is carried out at 85-110° C., preferably 90-95° C.; and/or
The step a1) is carried out under stirring; and/or
The reaction in the step a1) is carried out for 8-16 h; and/or
The catalyst in the step a1) is a copper catalyst, preferably, the copper catalyst is one or more of: metal copper, cuprous chloride, cuprous bromide, cuprous iodide, cuprous trifluoromethanesulfonate, cupric sulfate, cupric acetate, cupric trifluoromethylsulfonyl and cupric chloride, more preferably, the catalyst is cuprous trifluoromethanesulfonate, most preferably, the catalyst is a 1:1 complex of cuprous trifluoromethanesulfonate and benzonitrile; and/or
The solvent is one or more of: dichloroethane, dichloromethane and toluene, preferably, the solvent is dichloroethane;
More preferably, wherein:
The step a1) comprises:
a11) in a reactor, adding a compound of Formula (I) and a catalyst; and
a12) adding the solution obtained by dissolving the compound of Formula (II) in the solvent to the reactor and causing it to react; and/or
The step a2) comprises:
a21) distilling under vacuum the reaction product obtained in the step a1) and collecting a fraction to obtain the compound of Formula (III);
Still more preferably, wherein:
In the step a12), the mass ratio of the compound of Formula (II) and the solvent is 1:(1.5-2.5); and/or
The solvent in the step a12) is dichloroethane; and/or
The addition in the step a12) is carried out in a way of dropwise adding, preferably in a way of completing dropwise adding within 8-16 h; and/or
The distillation under vacuum in the step a21) is carried out by rotating the reactor; and/or
The collection of a fraction is carried out by collecting a fraction of 117-120° C./1 kPa.

In some embodiments, the step b) comprises:
b1) hydrolyzing the reaction mixture prepared by mixing a starting material, wherein the starting material contains:
a compound of Formula (III);
an inorganic base; and
a solvent; and
b2) separating from the reaction product obtained in the step b1) to obtain a compound of Formula (IV);
Preferably, wherein:
In the step b1), the weight ratio of the compound of Formula (III), the inorganic base and the solvent is 1: (0.35-0.5): (2-4), preferably 1:(0.4-0.5): (3-4), more preferably 1:0.44:3.43; and/or
In the step b1), the inorganic base is selected from sodium hydroxide and/or potassium hydroxide, preferably, the inorganic base is sodium hydroxide; and/or
In the step b1), the solvent is selected from water and/or alcohol, preferably, the solvent is a mixture of water and alcohol, more preferably, the solvent is a mixture of water and ethanol and the weight ratio of the water and the ethanol is 1:(1-2); and/or
The step b1) is carried out under stirring; and/or
The step b1) is carried out at 50-65° C.; and/or
The step b1) is carried out for 2-4 h;
More preferably, wherein:
The step b1) comprises: mixing the compound of Formula (III), the solvent and the inorganic base solution to set off a hydrolysis reaction; and/or
The step b2) comprises: removing the solvent from the reaction product obtained in the step b1) to obtain a compound of Formula (IV);
Still more preferably, wherein:
The solvent is a mixture of water and alcohol, preferably, the solvent is a mixture of water and ethanol, more preferably, the solvent is a 30-50 wt % ethanol aqueous solution; and/or
The inorganic base solution is an inorganic base aqueous solution, preferably, the inorganic base solution is a sodium hydroxide aqueous solution, more preferably is a 30-40 wt % sodium hydroxide aqueous solution.

In some embodiments, the step c) comprises:
c1) oxidizing the reaction mixture prepared by mixing a starting material, wherein the starting material contains:
a compound of Formula (IV);
TEMPO;
bicarbonate;
bromide;
hypochlorite; and
a solvent;
c2) adding a sulfite solid and/or a chlorite solid and/or a sulfite solution and/or a chlorite solution to the reaction product of the step c1);
c3) regulating the pH of the reaction system to 1-2; and
c4) separating from the product of the step c3) to obtain caronic acid;
Preferably, wherein:
The solvent in the step c1) is water; and/or
The step c1) is carried out at 20-30° C.; and/or
The step c1) is carried out at pH 8.5-10; and/or
The oxidation reaction in the step c1) is carried out for 10-16 h; and/or
The step c2) is carried out at 10-15° C.; and/or
The sulfite in the step c2) is sodium sulfite; and/or
The sulfite solution in the step c2) is a sodium sulfite aqueous solution; and/or
The chlorite in the step c2) is sodium chlorite; and/or
The chlorite solution in the step c2) is a sodium chlorite aqueous solution; and/or
The step c3) is realized by adding sulfuric acid; and/or
The step c1) and/or the step c2) and/or the step c3) are carried out under stirring;
More preferably, wherein:
The step c1) comprises:
c11) providing a solution of a compound of Formula (IV), regulating its pH to 8.5-10 and controlling its temperature at 10-15° C.;
c12) adding TEMPO, bicarbonate and bromide, then adding a hypochlorite solution, controlling the temperature at 10-15° C., and maintaining the pH of the reaction system at 8.5-10;
c13) raising the temperature to 20-30° C. and reacting for 10-16 h; and/or
The step c4) comprises:
c41) extracting the reaction product of the step c3) with an extraction agent to obtain an extract;
c42) removing the extraction agent from the extract to obtain a crude product; and
c43) crystalizing the crude product in a crystallization solvent and separating the solid to obtain caronic acid;
Still more preferably, wherein:
The solution of the compound of Formula (IV) in the step c11) is an aqueous solution of the compound of Formula (IV), preferably a 28-38 wt % aqueous solution; and/or
The regulation of pH to 8.5-10 in the step c12) is realized by adding sulfuric acid; and/or
The step c12) comprises first reducing the temperature to 10° C. after adding TEMPO, bicarbonate and bromide, and then adding a hypochlorite solution; and/or
The hypochlorite solution in the step c12) is a hypochlorite aqueous solution, preferably an 8-13 wt % hypochlorite aqueous solution, more preferably a 12 wt % hypochlorite aqueous solution; and/or
The maintenance of pH at 8.5-10 in the step c12) is realized by adding a liquid alkali; and/or
The extraction in the step c42) is carried out at 30-45° C.; and/or
The crystallization solvent in the step c43) contains:
water; and
alcohol, preferably methanol;
wherein, the weight ratio of water and alcohol is 1:(0.1-0.2).

In some embodiments, the caronic acid in the step s1) is prepared by the method comprising the following steps:
i) causing the reaction mixture prepared by mixing a starting material to react and separating the reaction product to obtain an organic phase product containing a compound of Formula (I′), wherein the starting material contains:
isopentenol;
acetic anhydride; and
carbonate;

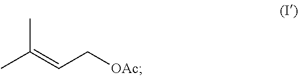

ii) causing the reaction mixture prepared by mixing a starting material to react and separating the reaction product to obtain an organic phase product containing a compound of Formula (II′), wherein the starting material contains:
glycine ethyl ester hydrochloride;
sodium nitrite;
a catalyst; and
a solvent;

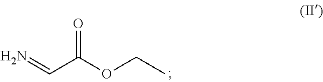

iii) mixing the organic phase product of the step i) and the organic phase product of the step ii), causing them to react and separating the reaction product to obtain a compound of Formula (III′),

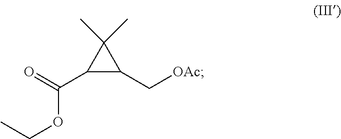

iv) hydrolyzing the compound of Formula (III′) to obtain a hydrolysis product containing the compound of Formula (IV),

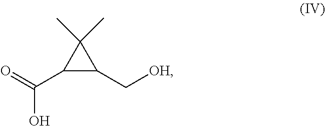

v) oxidizing the hydrolysis product of the step iv) and separating the reaction product to obtain caronic acid.

Preferably, the oxidation in the step v) is carried out using a TEMPO oxidation system.

Preferably, the TEMPO oxidation system comprises: TEMPO;
bicarbonate;
bromide; and
hypochlorite.

In some embodiments, wherein:
In the step i), the weight ratio of isopentenol, acetic anhydride and carbonate is 1:(1.25-1.65): (0.09-0.15), preferably 1:1.42:0.09; and/or
The carbonate in the step i) is potassium carbonate; and/or
The reaction in the step i) is carried out at 65° C.-75° C.; and/or
The reaction in the step i) is carried out for 5-8 h, preferably 6-7 h; and/or
In the organic phase product of the step i), the content of acetic acid is less than 0.6%, preferably less than 0.1%, preferably is realized by means of alkaline washing; and/or
In the step ii), the weight ratio of glycine ethyl ester hydrochloride, sodium nitrite, the catalyst and the solvent is 1:(0.5-0.65): (0.012-0.025): (3.1-5.0), preferably 1:0.6:0.015:4.6; and/or
The catalyst in the step ii) is formic acid and/or acetic acid, preferably acetic acid; and/or
The solvent in the step ii) is water and/or dichloroethane, preferably, the solvent is a mixture of water and dichloroethane, most preferably, the solvent is a mixture of water and dichloroethane and the weight ratio of the water and the dichloroethane is 1:(0.3-0.8), preferably 1:0.48; and/or
The reaction in the step ii) is carried out at 5-15° C.; and/or
The reaction in the step iii) is carried out for 8-16 h; and/or
In the step iii), the weight ratio of the organic phase product of the step i) and the organic phase product of the step ii) is 1:(3-3), and/or
the bicarbonate in the step v) is sodium bicarbonate; and/or
the bromide in the step v) is potassium bromide; and/or
the hypochlorite in the step v) is sodium hypochlorite; and/or
The weight ratio of the hydrolysis product of the step iv), the TEMPO, the bicarbonate, the potassium bromide and the hypochlorite in the step v) is 1:(0.01-0.05): (0.1-0.3): (0.01-0.03): (0.1-0.5), preferably 1:(0.008-0.01): (0.1-0.3): (0.01-0.03): (0.2-0.4), more preferably 1:0.009:0.19:0.016:0.33.

In some embodiments, wherein:
The step i) comprises:
i1) mixing isopentenol with carbonate;
i2) adding acetic anhydride to the mixture of the step i1) and causing them to react;
i3) adding water to the reactants of the step i2) and separating the organic phase to obtain the organic phase product;
Preferably, wherein:
The step i1) is carried out under stirring; and/or
The step i1) is carried out at 45-55° C., preferably 48-52° C.; and/or
The step i2) comprises dropwise adding acetic anhydride at 48-75° C. within 4-8 h and then stirring at 65-75° C. for 2-4 h; and/or
The step i3) comprises:
i31) cooling the reaction system to 20-35° C., preferably 28-32° C., dropwise adding water to the reaction system within 1-3 h, then adding water to the system at one time and continuing to stir at 20-35° C., preferably 28-32° C. for 0.5-2 h, preferably 30 min;
i32) letting stand and separating an organic phase to obtain the organic phase product.

In some embodiments, the step ii) comprises:
ii1) dissolving glycine ethyl ester hydrochloride in a solvent;
ii2) adding a solvent and a catalyst to the mixture obtained in the step ii1);
ii3) adding a sodium nitrite solution to the mixture obtained in the step ii2) and causing them to react; and
ii4) separating from the product obtained in the step ii3) to obtain the organic phase product;
Preferably, wherein:
In the step ii1), the weight ratio of the glycine ethyl ester hydrochloride and the solvent is 1:(1-2.4), preferably 1:2.1; and/or
The solvent in the step ii1) is water; and/or
In the step ii2), the weight ratio of the solvent and the catalyst is 1:(0.0085-0.015), preferably 1:0.0095; and/or
The solvent in the step ii2) is dichloroethane; and/or
The sodium nitrite solution in the step ii3) is a sodium nitrite aqueous solution, preferably a 20-50 wt % sodium nitrite aqueous solution, more preferably a 30-40 wt % sodium nitrite aqueous solution; and/or
The step ii3) comprises: dropwise adding a sodium nitrite aqueous solution, controlling the temperature at 5-15° C., completing the addition in about 4-8 h and then stirring again at 5-15° C. for 1-3 h; and/or
The step ii4) comprises: splitting the product obtained in the step ii3) into different phases, preferably using dichloroethane to extract the water phase and merging the organic phases to obtain the organic phase product.

In some embodiments, the step iii) comprises:
iii1) causing the reaction mixture prepared by mixing a starting material to react, wherein the starting material contains:
the organic phase product of the step i);
the organic phase product of the step ii); and
a catalyst; and
iii2) separating from the reaction product obtained in the step iii1) to obtain the compound of Formula (III′);
Preferably, wherein:
In the step iii1), the weight ratio of the organic phase product of the step i), the organic phase product of the step ii) and the catalyst is 1:(2-3): (0.005-0.015); and/or
The step iii1) is carried out at 85-110° C., preferably 90-95° C.; and/or
The step iii1) is carried out under stirring; and/or
The reaction in the step iii1) is carried out for 8-16 h; and/or
The catalyst in the step iii1) is a copper catalyst, preferably, the copper catalyst is one or more of: metal copper, cuprous chloride, cuprous bromide, cuprous iodide, cuprous trifluoromethanesulfonate, cupric sulfate, cupric acetate, cupric trifluoromethylsulfonyl and cupric chloride, more preferably, the catalyst is cuprous trifluoromethanesulfonate, most preferably, the catalyst is a 1:1 complex of cuprous trifluoromethanesulfonate and benzonitrile;
More preferably, wherein:
The step iii1) comprises:
iii11) in a reactor, adding the organic phase product of the step i) and a catalyst; and
iii12) adding the organic phase product of the step ii) to the reactor and causing them to react; and/or
The step iii2) comprises:
iii21) distilling under vacuum the reaction product obtained in the step iii1) and collecting a fraction to obtain the compound of Formula (III′);
Still more preferably, wherein:
In the organic phase product of the step i) in the step iii11), the concentration of the compound of Formula (I′) is 90-100 wt %; and/or
In the organic phase product of the step ii) in the step iii12), the concentration of the compound of Formula (II′) is 20-40 wt %, preferably 29 wt %; and/or
The addition in the step iii12) is carried out in a way of dropwise adding, preferably in a way of completing dropwise adding within 8-16 h; and/or
The distillation under vacuum in the step iii21) is carried out by rotating the reactor; and/or
The collection of a fraction in the step iii21) is carried out by collecting a fraction of 117-120° C./1 kPa.
In some embodiments, the step iv) comprises:
iv1) hydrolyzing the reaction mixture prepared by mixing a starting material, wherein the starting material contains:
a compound of Formula (III′);
an inorganic base; and
a solvent; and
iv2) obtaining a hydrolysis product containing the compound of Formula (IV) from the reaction product obtained in the step iv1).

Preferably, wherein:
In the step iv1), the weight ratio of the compound of Formula (III′), the inorganic base and the solvent is 1: (0.35-0.5): (2-4), preferably 1:(0.4-0.5): (3-4), more preferably 1:0.44:3.43; and/or
In the step iv1), the inorganic base is selected from sodium hydroxide and/or potassium hydroxide, preferably, the inorganic base is sodium hydroxide; and/or
In the step iv1), the solvent is selected from water and/or alcohol, preferably, the solvent is a mixture of water and alcohol, more preferably, the solvent is a mixture of water and ethanol and the weight ratio of the water and the ethanol is 1:(1-2); and/or
The step iv1) is carried out under stirring; and/or
The step iv1) is carried out at 50-65° C.; and/or
The step iv1) is carried out for 2-4 h;
More preferably, wherein:
The step iv1) comprises: mixing the compound of Formula (III′), the solvent and the inorganic base solution to set off a hydrolysis reaction; and/or
The step iv2) comprises: removing alcohol from the reaction product obtained in the step b1) to obtain an aqueous solution of the compound of Formula (IV), i.e., a hydrolysis product containing the compound of Formula (IV);
Still more preferably, wherein:
The solvent is a mixture of water and alcohol, preferably, the solvent is a mixture of water and ethanol, more preferably, the solvent is a 30-50 wt % ethanol aqueous solution; and/or
The inorganic base solution is an inorganic base aqueous solution, preferably, the inorganic base solution is a sodium hydroxide aqueous solution, more preferably is a 30-40 wt % sodium hydroxide aqueous solution; and/or
The content of the alcohol in the hydrolysis product containing the compound of Formula (IV) is less than 0.5 wt %, preferably less than 0.2 wt %.

In some embodiments, the step v) comprises:
v1) oxidizing the reaction mixture prepared by mixing a starting material, wherein the starting material contains:
a hydrolysis product containing the compound of Formula (IV);
TEMPO;
bicarbonate;
bromide; and
hypochlorite;
v2) adding a sulfite solid and/or a chlorite solid and/or a sulfite solution and/or a chlorite solution to the reaction product of the step v1);
v3) regulating the pH of the reaction system to 1-2; and
v4) separating from the product of the step v3) to obtain caronic acid;
Preferably, wherein:
The step v1) is carried out at 20-30° C.; and/or
The step v1) is carried out at pH 8.5-10; and/or
The oxidation reaction in the step v1) is carried out for 10-16 h; and/or
The step v2) is carried out at 10-15° C.; and/or
The sulfite in the step v2) is sodium sulfite; and/or
The sulfite solution in the step v2) is a sodium sulfite aqueous solution; and/or
The chlorite in the step v2) is sodium chlorite; and/or
The chlorite solution in the step v2) is a sodium chlorite aqueous solution; and/or
The step v3) is realized by adding sulfuric acid; and/or
The step v1) and/or the step v2) and/or the step v3) are carried out under stirring;
More preferably, wherein:
The step v1) comprises:
v11) providing a hydrolysis product containing the compound of Formula (IV), regulating its pH to 8.5-10 and controlling its temperature at 10-15° C.;
v12) adding TEMPO, bicarbonate and bromide, then adding a hypochlorite solution, controlling the temperature at 10-15° C., and maintaining the pH of the reaction system at 8.5-10;
v13) raising the temperature to 20-30° C. and reacting for 10-16 h; and/or The step v4) comprises:
v41) extracting the reaction product of the step v3) with an extraction agent to obtain an extract;
v42) removing the extraction agent from the extract to obtain a crude product; and
v43) crystalizing the crude product in a crystallization solvent and separating the solid to obtain caronic acid;
Still more preferably, wherein:
The hydrolysis product containing the compound of Formula (IV) in the step v11) is an aqueous solution of the compound of Formula (IV), preferably a 28-38 wt % aqueous solution; and/or
The regulation of pH to 8.5-10 in the step v12) is realized by adding sulfuric acid; and/or
The step v12) comprises first reducing the temperature to 10° C. after adding TEMPO, bicarbonate and bromide, and then adding a hypochlorite solution; and/or
The hypochlorite solution in the step v12) is a hypochlorite aqueous solution, preferably an 8-13 wt % hypochlorite aqueous solution, more preferably a 12 wt % hypochlorite aqueous solution; and/or
The maintenance of pH at 8.5-10 in the step v12) is realized by adding a liquid alkali; and/or
The extraction in the step v42) is carried out at 30-45° C.; and/or
The crystallization solvent in the step v43) contains:
water; and
alcohol, preferably methanol;
wherein, the weight ratio of water and alcohol is 1:(0.1-0.2).

It should be understood that the content described in this part is not intended to identify critical or significant features of the embodiments of the present disclosure, nor is it intended to limit the scope of the present disclosure. Other features of the present disclosure will become easily understood through the following description.

### DETAILED DESCRIPTION

Embodiments of the present invention will be described in more detail below. However, it should be understood that the present invention can be realized in various forms and should not be interpreted as limited to the embodiments set forth herein and on the contrary, these embodiments are provided to understand the present invention more thoroughly and completely. It should be understood that the embodiments of the present invention are used for exemplary purposes only and are not intended to limit the scope of protection of the present invention.

Unless otherwise defined, all technical and scientific terms used herein have the same meanings as those commonly understood by those skilled in the technical field of the present invention. In case of contradiction, the definitions herein shall prevail.

The terms “comprise,” “include,” “have,” “contain” or any other variation thereof as used herein are intended to cover non-exclusive inclusions.

When quantity, concentration, or any other value or parameter is expressed with a range, a preferred range, or a range delimited by a series of upper and lower preferred values, it should be understood that all the ranges formed by any pair of the upper or preferred value of any range and any lower or preferred value of any range are specifically disclosed, whether separately or not. For example, when the range of “1 to 5” is disclosed, the range described should be understood as including but not limited to ranges of “1 to 4,” “1 to 3,” “1 to 2,” “1-2 to 4-5” and “1-3 to 5.” When a numeric range is described herein, unless otherwise stated, the range is intended to include its end values and all integers and fractions within the range.

In addition, the indefinite articles “a” and “an” before an element or component herein are not restrictive to the quantitative requirements (i.e., the number of occurrences) of the element or component. Therefore, “a” or “an” should be understood as including one or at least one, and a singular form also includes a plural form for elements or components, unless the quantity stated is clearly intended to limit to the singular form.

Further, a large number of expressions will be mentioned in the following description, which are defined to have the following meanings.

“Caronic acid” refers to a compound of the following formula (CAS No. 497-42-7):

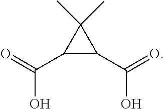

“TEMPO” refers to 2,2,6,6-tetramethylpiperidine oxide, an organic nitric oxide with CAS No. 2564-83-2.

“Catalyst” refers to a substance intended to increase the reaction rate.

“Extraction” refers to a unit operation of separating a mixture by using different solubility of the components in the system in the solvent.

“Crystallization” means that when a substance is in a non-equilibrium state, another phase will be precipitated in the form of crystal.

“Caronic anhydride” refers to a compound of the following formula (CAS No. 67911-21-1):

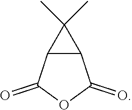

As described above, no methods are available in the prior art to obtain high-quality caronic anhydride with a higher yield, a higher purity and a lower cost more environmentally friendly.

### Method for Preparing Caronic Anhydride

In order to solve, at least in part, one or more of the foregoing problems and other potential problems, the first embodiment of the present disclosure proposes a method for preparing caronic anhydride, which comprises the following steps:
s1) cyclizing caronic acid to obtain crude caronic anhydride; and
s2) using isopropyl ether to recrystallize the crude caronic anhydride to obtain caronic anhydride.

There is no special limitation to the above step s1), which can be those commonly used in the art. Nevertheless, preferably, the step s1) comprises:
s11) causing the reaction mixture prepared by mixing a starting material to react, wherein the starting material contains:
caronic acid
a catalyst; and
a solvent; and
s12) distilling, preferably distilling under atmospheric pressure, the product obtained in the above step s11) to separate and obtain crude caronic anhydride.

There is no special limitation to the catalyst in the above step s11), which can be those commonly used in the art, such as sodium acetate, sodium hydroxide, sodium bicarbonate or sodium carbonate. Nevertheless, preferably, the catalyst is sodium acetate.

There is no special limitation to the above step s11), which can be those commonly used in the art. Nevertheless, preferably, the solvent is acetic anhydride.

There is no special limitation to the weight ratio of the caronic acid, the catalyst and the solvent in the step s11), which can be those commonly used in the art. Nevertheless, preferably, the weight ratio of the caronic acid, the catalyst and the solvent is 1:(0.01-0.05): (1-3), preferably 1:0.03:2.

There is no special limitation to the working temperature and time in the above step s11), which can be those commonly used in the art. Nevertheless, preferably, it is carried out at 130-140° C., preferably 135-138° C. for 1-5 h, preferably 2-4 h, more preferably 3 h.

There is no special limitation to the above step s12), which can be those commonly used in the art. Nevertheless, preferably, the step s12) comprises:
s121) distilling under atmospheric pressure at 140-145° C. the product obtained in the step s11) for 3-8 h to obtain a post-distillation product;
s122) heating and treating the post-distillation product, preferably in a high-temperature reactor, to collect a fraction of 165-200° C.;
s123) maintaining the temperature of the fraction obtained in the step s122) at 190-200° C. for 3-6 h to obtain crude caronic anhydride.

The above step s2) preferably comprises:
s21) dissolving the crude caronic anhydride in isopropyl ether at a high temperature, preferably 50-70° C., more preferably 55-60° C. to obtain a clear solution; and
s22) cooling the clear solution obtained in the above step s21) to a low temperature, preferably to 3-10° C., more preferably to 5-8° C. to precipitate a solid, i.e., caronic anhydride.

There is no special limitation to the caronic acid in the above step s1), which can be those commonly used in the art. Nevertheless, preferably, the caronic acid in the above step s1) can be prepared by one of the following two methods:

### First Method for Preparing Caronic Acid

In order to solve, at least in part, one or more of the foregoing problems and other potential problems, the second embodiment of the present disclosure proposes a method for preparing caronic acid, which comprises the following steps:
a) causing a compound of Formula (I) to react with a compound of Formula (II) to obtain a compound of Formula (III),

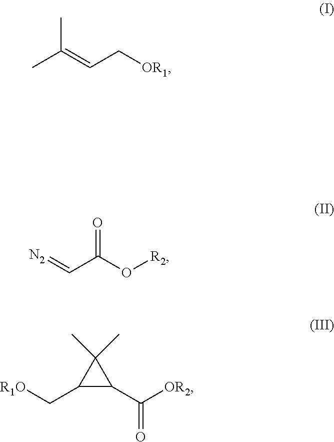

where the R₁ is a protecting group, preferably, the R₁ is selected from an ester protecting group, an alkyl ether protecting group and a silyl ether protecting group, more preferably, the ester protecting group is selected from acetyl, benzoyl and substituted benzoyl, the alkyl ether protecting group is selected from benzyl, triphenyl-methyl and tetrahydropyranyl, and the silyl ether protecting group is selected from trimethylsilyl and dimethyltert-butylsilyl, and most preferably, the protecting group is acetyl;

The R₂ is selected from: alkyl, cycloalkyl, aryl, alkylaryl, heterocyclyl and heteroaryl, preferably, the R₂ is selected from substituted or unsubstituted alkyl, more preferably, the R₂ is selected from unsubstituted alkyl, most preferably, the R₂ is ethyl;
b) hydrolyzing the compound of Formula (III) to obtain a compound of Formula (IV),

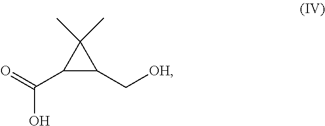

c) oxidizing the compound of Formula (IV) and obtaining caronic acid,
Preferably, the oxidation in the step c) is carried out using a TEMPO oxidation system.

Preferably, the TEMPO oxidation system comprises:
TEMPO;
bicarbonate;
bromide; and
hypochlorite.

There is no special limitation to the bicarbonate, which can be those commonly used in the art. Nevertheless, preferably, the bicarbonate is sodium bicarbonate. The present disclosure has unexpectedly discovered that the oxidation efficiency of the oxidation system can be effectively improved by adding bicarbonate.

There is no special limitation to the bromide, which can be those commonly used in the art. Nevertheless, preferably, the bromide is potassium bromide.

There is no special limitation to the hypochlorite, which can be those commonly used in the art. Nevertheless, preferably, the hypochlorite is sodium hypochlorite.

There is no special limitation to the weight ratio of the compound of Formula (IV), the TEMPO, the bicarbonate, the potassium bromide and the hypochlorite, which can be a ratio commonly used in the art. Nevertheless, preferably, in order to achieve a better oxidation effect, the weight ratio of the compound of Formula (IV), the TEMPO, the bicarbonate, the potassium bromide and the hypochlorite is 1:(0.01-0.05): (0.3-1): (0.01-0.1): (0.9-1.4), for example which can be 1:(0.01 or 0.02 or 0.03 or 0.04 or 0.05): (0.3 or 0.4 or 0.5 or 0.6 or 0.7 or 0.8 or 0.9 or 1): (0.01 or 0.02 or 0.03 or 0.04 or 0.05 or 0.06 or 0.07 or 0.08 or 0.09 or 0.1): (0.9 or 1.0 or 1.2 or 1.3 or 1.4), or any ratio among these ratios, most preferably 1:0.03:0.62:0.052:1.08.

There is no special limitation to the step a), which can be implemented in those manners commonly used in the art. Nevertheless, preferably, the step a) comprises:
a1) causing the reaction mixture prepared by mixing a starting material to react, wherein the starting material contains:
a compound of Formula (I);
a compound of Formula (II);
a catalyst; and
a solvent; and
a2) separating from the reaction product obtained in the step a1) to obtain the compound of Formula (III).

There is no special limitation to the weight ratio of the compound of Formula (I), the compound of Formula (II), the catalyst and the solvent in the step a1), which can be those commonly used in the art. Nevertheless, preferably, in the step a1), the weight ratio of the compound of Formula (I), the compound of Formula (II), the catalyst and the solvent is 1: (0.5-1.0): (0.005-0.015): (1.5-2.5), for example which can be 1: (0.5 or 0.6 or 0.7 or 0.8 or 0.9 or 1.0): (0.005 or 0.006 or 0.007 or 0.008 or 0.009 or 0.01 or 0.011 or 0.012 or 0.013 or 0.014 or 0.015): (1.5 or 1.6 or 1.7 or 1.8 or 1.9 or 2.0 or 2.1 or 2.2 or 2.3 or 2.4 or 2.5), or any ratio among these ratios, most preferably 1:0.74:0.008:1.8.

There is no special limitation to the temperature adopted in the step a1), which can be those commonly used in the art. Nevertheless, preferably, the step a1) is carried out at 85-110° C., preferably 90-95° C. For example, the temperature adopted can be 85, 86, 87, 88, 89, 90, 91, 92, 93, 94, 95, 96, 97, 98, 100, 101, 102, 103, 104, 105, 106, 107, 108, 109, 110° C. or any value between any two of these values.

Preferably, the step a1) is carried out under stirring.

There is no special limitation to the reaction time of the step a1), which can be those commonly used in the art. Nevertheless, preferably, the reaction in the step a1) is carried out for 8-16 h. For example, the number of hours adopted can be 8, 9, 10, 11, 12, 13, 14, 15, 16 h or any value between any two of these values.

There is no special limitation to the catalyst in the step a1), which can be those commonly used in the art. Nevertheless, preferably, the catalyst in the step a1) is a copper catalyst, preferably, the copper catalyst is one or more of: metal copper, cuprous chloride, cuprous bromide, cuprous iodide, cuprous trifluoromethanesulfonate, cupric sulfate, cupric acetate, cupric trifluoromethylsulfonyl and cupric chloride, more preferably, the catalyst is cuprous trifluoromethanesulfonate, most preferably, the catalyst is a 1:1 complex of cuprous trifluoromethanesulfonate and benzonitrile.

There is no special limitation to the solvent, which can be those commonly used in the art. Nevertheless, preferably, the solvent is one or more of: dichloroethane, dichloromethane and toluene, preferably, the solvent is dichloroethane.

Preferably, the step a1) comprises:
a11) in a reactor, adding a compound of Formula (I) and a catalyst; and
a12) adding the solution obtained by dissolving the compound of Formula (II) in the solvent to the reactor and causing it to react; and/or
The step a2) comprises:
a21) distilling under vacuum the reaction product obtained in the step a1) and collecting a fraction to obtain the compound of Formula (III).

There is no special limitation to the mass ratio of the compound of Formula (II) and the solvent in the step a12), which can be those commonly used in the art. Nevertheless, preferably, in the step a12), the mass ratio of the compound of Formula (II) and the solvent is 1:1.5, 1:1.6, 1:1.7, 1:1.8, 1:1.9, 1:2.0, 1:2.1, 1:2.2, 1:2.3, 1:2.4, 1:2.5, 1:2.6 or any value between any two of these values.

There is no special limitation to the solvent in the step a12), which can be those commonly used in the art. Nevertheless, preferably, the solvent in the step a12) is dichloroethane.

There is no special limitation to the way of addition in the step a12), which can be those commonly used in the art. Nevertheless, preferably, the addition in the step a12) is carried out in a way of dropwise adding, preferably in a way of completing dropwise adding within 8-16 h.

There is no special limitation to the manner in which the distillation under vacuum in the step a21) is implemented, which can be those commonly used in the art. Nevertheless, preferably, the distillation under vacuum in the step a21) is carried out by rotating the reactor.

There is no special limitation to the conditions under which the collection of a fraction in the step a21) is implemented, which can be those commonly used in the art. Nevertheless, preferably, the collection of a fraction is carried out by collecting a fraction of 117-120° C./1 kPa.

There is no special limitation to the step b), which can be implemented in those manners commonly used in the art. Nevertheless, preferably, the step b) comprises:
b1) hydrolyzing the reaction mixture prepared by mixing a starting material, wherein the starting material contains:
a compound of Formula (III);
an inorganic base; and
a solvent; and
b2) separating from the reaction product obtained in the step b1) to obtain a compound of Formula (IV).

There is no special limitation to the weight ratio of the compound of Formula (III), the inorganic base and the solvent in the step b1), which can be those commonly used in the art. Nevertheless, preferably, in the step b1), the weight ratio of the compound of Formula (III), the inorganic base and the solvent is 1: (0.35-0.5): (2-4), preferably 1:(0.4-0.5): (3-4), more preferably 1:0.44:3.43, for example which can be 1: (0.35 or 0.36 or 0.37 or 0.38 or 0.39 or 0.40 or 0.41 or 0.42 or 0.43 or 0.44 or 0.45 or 0.46 or 0.47 or 0.48 or 0.49 or 0.5): (2 or 3 or 4), or any ratio among these ratios.

There is no special limitation to the inorganic base in the step b1), which can be those commonly used in the art. Nevertheless, preferably, the inorganic base is selected from sodium hydroxide and/or potassium hydroxide, more preferably, the inorganic base is sodium hydroxide; and/or

There is no special limitation to the solvent in the step b1), which can be those commonly used in the art. Nevertheless, preferably, the solvent is selected from water and/or alcohol, more preferably, the solvent is a mixture of water and alcohol, still more preferably, the solvent is a mixture of water and ethanol and the weight ratio of the water and the ethanol is 1:(1-2).

Preferably, the step b1) is carried out under stirring.

There is no special limitation to the temperature adopted in the step b1), which can be those commonly used in the art. Nevertheless, preferably, the step b1) is carried out at 50-65° C. For example, the temperature adopted can be 50, 51, 52, 53, 54, 55, 56, 57, 58, 59, 60, 61, 62, 63, 64, 65° C. or any value between any two of these values.

There is no special limitation to the time in the step b1), which can be those commonly used in the art. Nevertheless, preferably, the step b1) is carried out for 2-4 h. For example, it can be 2, 3, 4 h or any value between any two of these values.

Preferably, the step b1) comprises: mixing the compound of Formula (III), the solvent and the inorganic base solution to set off a hydrolysis reaction; and/or

The step b2) comprises: removing the solvent from the reaction product obtained in the step b1) to obtain a compound of Formula (IV);

Still more preferably, the solvent is a mixture of water and alcohol, preferably, the solvent is a mixture of water and ethanol, more preferably, the solvent is a 30-50 wt % ethanol aqueous solution; and/or

The inorganic base solution is an inorganic base aqueous solution, preferably, the inorganic base solution is a sodium hydroxide aqueous solution, more preferably is a 30-40 wt % sodium hydroxide aqueous solution.

There is no special limitation to the step c), which can be implemented in those manners commonly used in the art. Nevertheless, preferably, the step c) comprises:
c1) oxidizing the reaction mixture prepared by mixing a starting material, wherein the starting material contains:
a compound of Formula (IV);
TEMPO;
bicarbonate;
bromide;
hypochlorite; and
a solvent;
c2) adding a sulfite solid and/or a chlorite solid and/or a sulfite solution and/or a chlorite solution to the reaction product of the step c1);
c3) regulating the pH of the reaction system to 1-2; and
c4) separating from the product of the step c3) to obtain caronic acid.

There is no special limitation to the solvent in the step c1), which can be those commonly used in the art. Nevertheless, preferably, the solvent in the step c1) is water.

There is no special limitation to the temperature adopted in the step c1), which can be those commonly used in the art. Nevertheless, preferably, the step c1) is carried out at 20-30° C. For example, the temperature adopted can be 20, 21, 22, 23, 24, 25, 26, 27, 28, 29, 30° C. or any value between any two of these values.

There is no special limitation to the pH adopted in the step c1), which can be those commonly used in the art. Nevertheless, preferably, the step c1) is carried out at pH 8.5-10. For example, the pH adopted can be 8.5, 8.6, 8.7, 8.8, 8.9, 9.0, 9.1, 9.2, 9.3, 9.4, 9.5, 9.6, 9.7, 9.8, 9.9, 10 or any value between any two of these values.

There is no special limitation to the time for which the oxidation reaction in the step c1) is carried out, which can be those commonly used in the art. Nevertheless, preferably, the oxidation reaction in the step c1) is carried out for 10-16 h. For example, the number of hours adopted can be 10, 11, 12, 13, 14, 15, 16 h or any value between any two of these values.

There is no special limitation to the temperature adopted in the step c2), which can be those commonly used in the art. Nevertheless, preferably, the step c2) is carried out at 10-15° C. For example, the temperature adopted can be 10, 11, 12, 13, 14, 15° C. or any value between any two of these values.

There is no special limitation to the sulfite in the step c2), which can be those commonly used in the art. Nevertheless, preferably, the sulfite in the step c2) is sodium sulfite.

There is no special limitation to the sulfite solution in the step c2), which can be those commonly used in the art. Nevertheless, preferably, the sulfite solution in the step c2) is a sodium sulfite aqueous solution.

There is no special limitation to the chlorite in the step c2), which can be those commonly used in the art. Nevertheless, preferably, the chlorite in the step c2) is sodium chlorite.

There is no special limitation to the sulfite solution in the step c2), which can be those commonly used in the art. Nevertheless, preferably, the chlorite solution in the step c2) is a sodium chlorite aqueous solution.

There is no special limitation to the pH regulated in the step c3), which can be those commonly used in the art. Nevertheless, preferably, the step c3) is realized by adding sulfuric acid.

Preferably, the step c1) and/or the step c2) and/or the step c3) are carried out under stirring.

Preferably, the step c1) comprises:
c11) providing a solution of the compound of Formula (IV), regulating its pH to 8.5-10 (for example, it can be 8.5, 8.6, 8.7, 8.8, 8.9, 9.0, 9.1, 9.2, 9.3, 9.4, 9.5, 9.6, 9.7, 9.8, 9.9, 10 or any value between any two of these values), and controlling the temperature at 10-15° C. (such as: 10, 11, 12, 13, 14, 15° C. or any value between any two of these values);
c12) adding TEMPO, bicarbonate and bromide, then adding a hypochlorite solution, controlling the temperature at 10-15° C. (such as: 10, 11, 12, 13, 14, 15° C. or any value between any two of these values), and maintaining the pH of the reaction system at 8.5-10 (for example, it can be 8.5, 8.6, 8.7, 8.8, 8.9, 9.0, 9.1, 9.2, 9.3, 9.4, 9.5, 9.6, 9.7, 9.8, 9.9, 10 or any value between any two of these values);
c13) raising the temperature to 20-30° C. (such as: 20, 21, 22, 23, 24, 25, 26, 27, 28, 29, 30° C. or any value between any two of these values), reacting for 10-16 h (for example, 10, 11, 12, 13, 14, 15, 16 h or any value between any two of these values); and/or
The step c4) comprises:
c41) extracting the reaction product of the step c3) with an extraction agent to obtain an extract;
c42) removing the extraction agent from the extract to obtain a crude product; and
c43) crystalizing the crude product in a crystallization solvent and separating the solid to obtain caronic acid.

Preferably, the solution of the compound of Formula (IV) in the step c11) is an aqueous solution of the compound of Formula (IV), preferably a 28-38 wt % aqueous solution. For example, the concentration of the aqueous solution can be 28, 29, 30, 31, 32, 33, 34, 35, 36, 37, 38 wt % or any value between any two of these values; and/or
The regulation of pH to 8.5-10 in the step c12) is realized by adding sulfuric acid; and/or
The step c12) comprises first reducing the temperature to 10° C. after adding TEMPO, bicarbonate and bromide, and then adding a hypochlorite solution; and/or
The hypochlorite solution in the step c12) is a hypochlorite aqueous solution, preferably an 8-13 wt % hypochlorite aqueous solution, more preferably a 12 wt % hypochlorite aqueous solution. For example, the concentration of the hypochlorite aqueous solution can be 8, 9, 10, 11, 12, 13 wt % or any value between any two of these values; and/or
The maintenance of pH at 8.5-10 in the step c12) is realized by adding a liquid alkali; and/or
The extraction in the step c42) is carried out at 30-45° C. For example, the temperature can be 30, 31, 32, 33, 34, 35, 36, 37, 38, 39, 40, 41, 42, 43, 44, 45° C. or any value between any two of these values; and/or
The crystallization solvent in the step c43) contains:
water; and
alcohol, preferably methanol;
wherein, the weight ratio of water and alcohol is 1:(0.1-0.2).

### Second Method for Preparing Caronic Acid

In order to solve, at least in part, one or more of the foregoing problems and other potential problems, the third embodiment of the present disclosure proposes a method for preparing caronic acid, which comprises the following steps:
i) causing the reaction mixture prepared by mixing a starting material to react and separating the reaction product to obtain an organic phase product containing a compound of Formula (I′), wherein the starting material contains:
isopentenol;
acetic anhydride; and
carbonate;

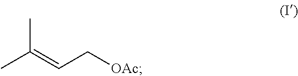

ii) causing the reaction mixture prepared by mixing a starting material to react and separating the reaction product to obtain an organic phase product containing a compound of Formula (II′), wherein the starting material contains:
glycine ethyl ester hydrochloride;
sodium nitrite;
a catalyst; and
a solvent;

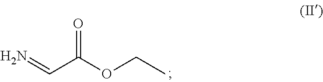

iii) mixing the organic phase product of the step i) and the organic phase product of the step ii), causing them to react and separating the reaction product to obtain a compound of Formula (III′),

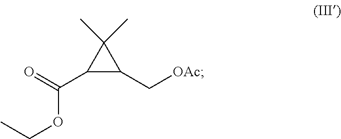

iv) hydrolyzing the compound of Formula (III′) to obtain a hydrolysis product containing the compound of Formula (IV),

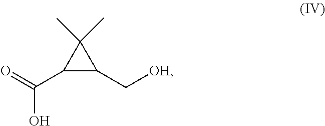

v) oxidizing the hydrolysis product of the step iv) and separating the reaction product to obtain caronic acid.

Preferably, the oxidation in the step v) is carried out using a TEMPO oxidation system.

Preferably, the TEMPO oxidation system comprises:
TEMPO;
bicarbonate;
bromide; and
hypochlorite.

There is no special limitation to the weight ratio of isopentenol, acetic anhydride and carbonate in the step i), which can be those commonly used in the art. Nevertheless, preferably, in the step i), the weight ratio of isopentenol, acetic anhydride and carbonate is 1:(1.25-1.65): (0.09-0.15), preferably 1:1.42:0.09. For example, the weight ratio of isopentenol, acetic anhydride and carbonate can be 1:(1.25 or 1.35 or 1.45 or 1.55 or 1.65): (0.09 or 0.1 or 0.11 or 0.12 or 0.13 or 0.14 or 0.15), or any ratio among these ratios.

There is no special limitation to the carbonate in the step i), which can be those commonly used in the art. Nevertheless, preferably, the carbonate in the step i) is potassium carbonate.

There is no special limitation to the temperature in the step i), which can be those commonly used in the art. Nevertheless, preferably, the reaction in the step i) is carried out at 65° C.-75° C. For example, the temperature adopted can be 65, 66, 67, 68, 69, 70, 71, 72, 73, 74, 75° C. or any value between any two of these values.

There is no special limitation to the reaction time of the step i), which can be those commonly used in the art. Nevertheless, preferably, the reaction in the step i) is carried out for 5-8 h, preferably 6-7 h. For example, it can be 5, 6, 7, 8 h or any value between any two of these values.

Preferably, in the organic phase product of the step i), the content of acetic acid is less than 0.6%, preferably less than 0.1%, preferably is realized by means of alkaline washing. For example, the content of acetic acid can be controlled less than 0.6%, less than 0.5%, less than 0.4%, less than 0.3%, less than 0.2%, less than 0.1% or even less. By controlling the content of acetic acid in a lower range, the toxic effect on the catalyst can be effectively reduced and the reaction degree can be improved.

There is no special limitation to the weight ratio of glycine ethyl ester hydrochloride, sodium nitrite, the catalyst and the solvent in the step ii), which can be those commonly used in the art. Nevertheless, preferably, in the step ii), the weight ratio of glycine ethyl ester hydrochloride, sodium nitrite, the catalyst and the solvent is 1:(0.5-0.65): (0.012-0.025): (3.1-5.0), preferably 1:0.6:0.015:4.6. For example, the weight ratio can be 1:(0.5 or 0.51 or 0.52 or 0.53 or 0.54 or 0.55 or 0.56 or 0.57 or 0.58 or 0.59 or 0.6 or 0.61 or 0.62 or 0.63 or 0.64 or 0.65): (0.012 or 0.013 or 0.014 or 0.015 or 0.016 or 0.017 or 0.018 or 0.019 or 0.020 or 0.021 or 0.022 or 0.023 or 0.024 or 0.025): (3.1 or 3.2 or 3.3 or 3.4 or 3.5 or 3.6 or 3.7 or 3.8 or 3.9 or 4.0 or 4.1 or 4.2 or 4.3 or 4.4 or 4.5 or 4.6 or 4.7 or 4.8 or 4.9 or 5.0), or any ratio among these ratios.

There is no special limitation to the catalyst in the step ii), which can be those commonly used in the art, such as: sulfuric acid. Nevertheless, preferably, the catalyst in the step ii) is formic acid and/or acetic acid, preferably acetic acid.

There is no special limitation to the solvent in the step ii), which can be those commonly used in the art. Nevertheless, preferably, the solvent in the step ii) is water and/or dichloroethane, more preferably, the solvent is a mixture of water and dichloroethane, most preferably, the solvent is a mixture of water and dichloroethane and the weight ratio of the water and the dichloroethane is 1:(0.3-0.8), preferably 1:0.48. For example, the weight ratio can be 1:0.3, 1:0.4, 1:0.48, 1:0.5, 1:0.6, 1:0.7, 1:0.8 or any ratio among these ratios.

There is no special limitation to the reaction temperature in the step ii), which can be those commonly used in the art. Nevertheless, preferably, the reaction in the step ii) is carried out at 5-15° C. For example, the temperature adopted can be 5, 6, 7, 8, 9, 10, 11, 12, 13, 14, 15° C. or any value between any two of these values.

There is no special limitation to the reaction time of the step iii), which can be those commonly used in the art. Nevertheless, preferably, the reaction in the step iii) is carried out for 8-16 h. For example, the reaction time can be 8, 9, 10, 11, 12, 13, 14, 15, 16 h or any value between any two of these values.

In the step iii), there is no special limitation to the weight ratio of the organic phase product of the step i) and the organic phase product of the step ii), which can be those commonly used in the art. Nevertheless, preferably, in the step iii), the weight ratio of the organic phase product of the step i) and the organic phase product of the step ii) is 1:(2-3). For example, it can be 1:0.5, 1:0.6, 1:0.7, 1:0.8, 1:0.9, 1:1.0 or any value between any two of these values.

There is no special limitation to the bicarbonate in the step v), which can be those commonly used in the art. Nevertheless, preferably, the bicarbonate in the step v) is sodium bicarbonate.

There is no special limitation to the bromide in the step v), which can be those commonly used in the art. Nevertheless, preferably, the bromide in the step v) is potassium bromide.

There is no special limitation to the hypochlorite in the step v), which can be those commonly used in the art. Nevertheless, preferably, the hypochlorite in the step v) is sodium hypochlorite.

The weight ratio of the hydrolysis product of the step iv), the TEMPO, the bicarbonate, the potassium bromide and the hypochlorite in the step v) is 1:(0.01-0.05): (0.1-0.3): (0.01-0.03): (0.1-0.5), preferably 1:(0.008-0.01): (0.1-0.3): (0.01-0.03): (0.2-0.4), more preferably 1:0.009:0.19:0.016:0.33.

Preferably, the step i) comprises:
i1) mixing isopentenol with carbonate;
i2) adding acetic anhydride to the mixture of the step i1) and causing them to react;
i3) adding water to the reactants of the step i2) and separating the organic phase to obtain the organic phase product.

Preferably, the step i1) is carried out under stirring.

There is no special limitation to the temperature adopted in the step i1), which can be those commonly used in the art. Nevertheless, preferably, the step i1) is carried out at 45-55° C., preferably 48-52° C. For example, the temperature adopted can be 45, 46, 47, 48, 49, 50, 51, 52, 53, 54, 55° C. or any value between any two of these values.

Preferably, the step i2) comprises dropwise adding acetic anhydride at 48-75° C. (such as: 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75° C. or any value between any two of these values) within 4-8 h (such as: 4, 5, 6, 7, 8 h or any value between any two of these values) and then stirring at 65-75° C. (65, 66, 67, 68, 69, 70, 71, 72, 73, 74, 75° C. or any value between any two of these values) for 2-4 h (such as: 2, 3, 4 h or any value between any two of these values).

Preferably, the step i3) comprises:
i31) cooling the reaction system to 20-35° C. (such as: 20, 21, 22, 23, 24, 25, 26, 27, 28, 29, 30, 31, 32, 33, 34, 35° C. or any value between any two of these values), preferably 28-32° C., dropwise adding water to the reaction system within 1-3 h (such as: 1, 2, 3 h or any value between any two of these values), then adding water to the system at one time and continuing to stir at 20-35° C. (such as: 20, 21, 22, 23, 24, 25, 26, 27, 28, 29, 30, 31, 32, 33, 34, 35° C. or any value between any two of these values), preferably 28-32° C. for 0.5-2 h (such as: 0.5, 1, 2 h or any value between any two of these values), preferably 30 min;
i32) letting stand and separating an organic phase to obtain the organic phase product.

Preferably, the step ii) comprises:
ii1) dissolving glycine ethyl ester hydrochloride in a solvent;
ii2) adding a solvent and a catalyst to the mixture obtained in the step ii1);
ii3) adding a sodium nitrite solution to the mixture obtained in the step ii2) and causing them to react; and
ii4) separating from the product obtained in the step ii3) to obtain the organic phase product.

There is no special limitation to the weight ratio of the glycine ethyl ester hydrochloride and the solvent in the step ii1), which can be those commonly used in the art. Nevertheless, preferably, in the step ii1), the weight ratio of the glycine ethyl ester hydrochloride and the solvent is 1:(1-2.4), preferably 1:2.1. For example, the weight ratio can be 1:1, 1:1.1, 1:2, 1:1.3, 1:4, 1:1.5, 1:1.6, 1:1.7, 1:1.8, 1:1.9, 1:2.0, 1:2.1 or any value between any two of these values.

There is no special limitation to the solvent in the step ii1), which can be those commonly used in the art. Nevertheless, preferably, the solvent in the step ii1) is water.

There is no special limitation to the weight ratio of the solvent and the catalyst in the step ii2), which can be those commonly used in the art. Nevertheless, preferably, in the step ii2), the weight ratio of the solvent and the catalyst is 1:(0.0085-0.015), preferably 1:0.0095. For example, the weight ratio, which can be adopted, is 1:0.0085, 1:0.0090, 1:0.0095, 1:0.01, 1:0.015 or any value between any two of these values.

There is no special limitation to the solvent in the step ii2), which can be those commonly used in the art. Nevertheless, preferably, the solvent in the step ii2) is dichloroethane.

There is no special limitation to the sodium nitrite in the step ii3), which can be those commonly used in the art. Nevertheless, preferably, the sodium nitrite solution in the step ii3) is a sodium nitrite aqueous solution, preferably a 20-50 wt % sodium nitrite aqueous solution. For example, the concentration of the sodium nitrite aqueous solution can be 20, 21, 22, 23, 24, 25, 26, 27, 28, 29, 30, 30, 31, 32, 33, 34, 35, 36, 37, 38, 39, 40, 41, 42, 43, 44, 45, 46, 47, 48, 49, 50 wt % or any value between any two of these values.

Preferably, the step ii3) comprises: dropwise adding a sodium nitrite aqueous solution, controlling the temperature at 5-15° C. (such as: 5, 6, 7, 8, 9, 10, 11, 12, 13, 14, 15° C. or any value between any two of these values) within about 4-8 h (such as: 4, 5, 6, 7, 8 or any value between any two of these values) and stirring again at 5-15° C. (such as: 5, 6, 7, 8, 9, 10, 11, 12, 13, 14, 15° C. or any value between any two of these values) for 1-3 h (such as: 1, 2, 3 h or any value between any two of these values); and/or

The step ii4) comprises: splitting the product obtained in the step ii3) into different phases, preferably using dichloroethane to extract the water phase and merging the organic phases to obtain the organic phase product.

Preferably, the step iii) comprises:
iii1) causing the reaction mixture prepared by mixing a starting material to react, wherein the starting material contains:
the organic phase product of the step i);
the organic phase product of the step ii); and
a catalyst; and
iii2) separating from the reaction product obtained in the step iii1) to obtain the compound of Formula (III′).

In the step iii1), there is no special limitation to the weight ratio of the organic phase product of the step i), the organic phase product of the step ii) and the catalyst, which can be those commonly used in the art. Nevertheless, preferably, in the step iii1), the weight ratio of the organic phase product of the step i), the organic phase product of the step ii) and the catalyst is 1:(2-3): (0.005-0.015). For example, the weight ratio can be 1:(2 or 3): (0.005 or 0.006 or 0.007 or 0.008 or 0.009 or 0.01 or 0.011 or 0.012 or 0.013 or 0.014 or 0.015) or any ratio among these ratios.

There is no special limitation to the temperature adopted in the step iii1), which can be those commonly used in the art. Nevertheless, preferably, the step iii1) is carried out at 85-110° C., preferably 90-95° C. For example, the temperature adopted can be 85, 86, 87, 88, 89, 90, 91, 92, 93, 94, 95, 96, 97, 98, 100, 101, 102, 103, 104, 105, 106, 107, 108, 109, 110° C. or any value between any two of these values.

Preferably, the step iii1) is carried out under stirring.

There is no special limitation to the reaction time in the step iii1), which can be those commonly used in the art. Nevertheless, preferably, the reaction in the step iii1) is carried out for 8-16 h. For example, the number of hours adopted can be 8, 9, 10, 11, 12, 13, 14, 15, 16 h or any value between any two of these values.

There is no special limitation to the catalyst in the step iii1), which can be those commonly used in the art. Nevertheless, preferably, the catalyst in the step iii1) is a copper catalyst, preferably, the copper catalyst is one or more of: metal copper, cuprous chloride, cuprous bromide, cuprous iodide, cuprous trifluoromethanesulfonate, cupric sulfate, cupric acetate, cupric trifluoromethylsulfonyl and cupric chloride, more preferably, the catalyst is cuprous trifluoromethanesulfonate, most preferably, the catalyst is a 1:1 complex of cuprous trifluoromethanesulfonate and benzonitrile.

Preferably, the step iii1) comprises:
iii11) in a reactor, adding the organic phase product of the step i) and a catalyst; and
iii12) adding the organic phase product of the step ii) to the reactor and causing them to react; and/or
The step iii2) comprises:
iii21) distilling under vacuum the reaction product obtained in the step iii1) and collecting a fraction to obtain the compound of Formula (III′).

In the step iii11), there is no special limitation to the concentration of the compound of Formula (I′) in the organic phase product of the step i), which can be those commonly used in the art. Nevertheless, preferably, the concentration is 90-100 wt %. For example, the concentration can be 90, 91, 92, 93, 94, 95, 96, 97, 98, 99, 100 wt % or any value between any two of these values.

In the step iii12), there is no special limitation to the concentration of the compound of Formula (II′) in the organic phase product of the step ii), which can be those commonly used in the art. Nevertheless, preferably, the concentration of the organic phase product of the step ii) in the step iii12) is 20-40 wt %. For example, the concentration can be 20, 21, 22, 23, 24, 25, 26, 27, 28, 29, 30, 31, 32, 33, 34, 35, 36, 37, 38, 39, 40 wt % or any value between any two of these values.

There is no special limitation to the way of addition in the step iii12), which can be those commonly used in the art. Nevertheless, preferably, the addition in the step iii12) is carried out in a way of dropwise adding, preferably in a way of completing dropwise adding within 8-16 h.

There is no special limitation to the manner in which the distillation under vacuum in the step iii21) is implemented, which can be those commonly used in the art. Nevertheless, preferably, the distillation under vacuum in the step iii21) is carried out by rotating the reactor.

There is no special limitation to the conditions under which a fraction is collected in the step iii21), which can be those commonly used in the art. Nevertheless, preferably, the collection of a fraction is carried out by collecting a fraction of 117-120° C./1 kPa.

There is no special limitation to the step iv), which can be implemented in those manners commonly used in the art. Nevertheless, preferably, the step iv) comprises:
iv1) hydrolyzing the reaction mixture prepared by mixing a starting material, wherein the starting material contains:
a compound of Formula (III′);
an inorganic base; and
a solvent; and
iv2) obtaining a hydrolysis product containing the compound of Formula (IV) from the reaction product obtained in the step iv1).

There is no special limitation to the weight ratio of the compound of Formula (III′), the inorganic base and the solvent in the step iv1), which can be those commonly used in the art. Nevertheless, preferably, in the step iv1), the weight ratio of the compound of Formula (III′), the inorganic base and the solvent is 1: (0.35-0.5): (2-4), preferably 1:(0.4-0.5): (3-4), more preferably 1:0.44:3.43, for example which can be 1: (0.35 or 0.36 or 0.37 or 0.38 or 0.39 or 0.40 or 0.41 or 0.42 or 0.43 or 0.44 or 0.45 or 0.46 or 0.47 or 0.48 or 0.49 or 0.5): (2 or 3 or 4), or any ratio among these ratios.

There is no special limitation to the inorganic base in the step iv1), which can be those commonly used in the art. Nevertheless, preferably, the inorganic base is selected from sodium hydroxide and/or potassium hydroxide, more preferably, the inorganic base is sodium hydroxide; and/or
There is no special limitation to the solvent in the step iv1), which can be those commonly used in the art. Nevertheless, preferably, the solvent is selected from water and/or alcohol, more preferably, the solvent is a mixture of water and alcohol, still more preferably, the solvent is a mixture of water and ethanol and the weight ratio of the water and the ethanol is 1:(1-2).

Preferably, the step iv1) is carried out under stirring.

There is no special limitation to the temperature adopted in the step iv1), which can be those commonly used in the art. Nevertheless, preferably, the step b1) is carried out at 50-65° C. For example, the temperature adopted can be 50, 51, 52, 53, 54, 55, 56, 57, 58, 59, 60, 61, 62, 63, 64, 65° C. or any value between any two of these values.

There is no special limitation to the time in the step iv1), which can be those commonly used in the art. Nevertheless, preferably, the step b1) is carried out for 2-4 h. For example, it can be 2, 3, 4 h or any value between any two of these values.

Preferably, the step iv1) comprises: mixing the compound of Formula (III′), the solvent and the inorganic base solution to set off a hydrolysis reaction; and/or

The step iv2) comprises: removing alcohol from the reaction product obtained in the step b1) to obtain an aqueous solution of the compound of Formula (IV), i.e., a hydrolysis product containing the compound of Formula (IV).

Still more preferably, the solvent is a mixture of water and alcohol, preferably, the solvent is a mixture of water and ethanol, more preferably, the solvent is a 30-50 wt % ethanol aqueous solution; and/or
The inorganic base solution is an inorganic base aqueous solution, preferably, the inorganic base solution is a sodium hydroxide aqueous solution, more preferably is a 30-40 wt % sodium hydroxide aqueous solution; and/or
The content of the alcohol in the hydrolysis product containing the compound of Formula (IV) is less than 0.5 wt %, preferably less than 0.2 wt %. For example, it can be less than 0.5 wt %, less than 0.4 wt %, less than 0.3 wt %, less than 0.2 wt % or even less.

Preferably, the step v) comprises:
v1) oxidizing the reaction mixture prepared by mixing a starting material, wherein the starting material contains:
a hydrolysis product containing the compound of Formula (IV);
TEMPO;
bicarbonate;
bromide; and
hypochlorite;
v2) adding a sulfite solid and/or a chlorite solid and/or a sulfite solution and/or a chlorite solution to the reaction product of the step v1);
v3) regulating the pH of the reaction system to 1-2; and
v4) separating from the product of the step v3) to obtain caronic acid.

There is no special limitation to the temperature adopted in the step v1), which can be those commonly used in the art. Nevertheless, preferably, the step v1) is carried out at 20-30° C. For example, the temperature adopted can be 20, 21, 22, 23, 24, 25, 26, 27, 28, 29, 30° C. or any value between any two of these values.

There is no special limitation to the pH adopted in the step v1), which can be those commonly used in the art. Nevertheless, preferably, the step v1) is carried out at pH 8.5-10. For example, the pH adopted can be 8.5, 8.6, 8.7, 8.8, 8.9, 9.0, 9.1, 9.2, 9.3, 9.4, 9.5, 9.6, 9.7, 9.8, 9.9, 10 or any value between any two of these values.

There is no special limitation to the time for which the oxidation reaction in the step c1) is carried out, which can be those commonly used in the art. Nevertheless, preferably, the oxidation reaction in the step c1) is carried out for 10-16 h. For example, the number of hours adopted can be 10, 11, 12, 13, 14, 15, 16 h or any value between any two of these values.

There is no special limitation to the temperature adopted in the step v2), which can be those commonly used in the art. Nevertheless, preferably, the step v2) is carried out at 10-15° C. For example, the temperature adopted can be 10, 11, 12, 13, 14, 15° C. or any value between any two of these values.

There is no special limitation to the sulfite in the step v2), which can be those commonly used in the art. Nevertheless, preferably, the sulfite in the step v2) is sodium sulfite.

There is no special limitation to the sulfite solution in the step v2), which can be those commonly used in the art. Nevertheless, preferably, the sulfite solution in the step v2) is a sodium sulfite aqueous solution.

There is no special limitation to the chlorite in the step v2), which can be those commonly used in the art. Nevertheless, preferably, the chlorite in the step v2) is sodium chlorite.

There is no special limitation to the chlorite solution in the step v2), which can be those commonly used in the art. Nevertheless, preferably, the chlorite solution in the step v2) is a sodium chlorite aqueous solution.

There is no special limitation to the regulation of pH in the step v3), which can be those commonly used in the art. Nevertheless, preferably, the step v3) is realized by adding sulfuric acid.

Preferably, the step v1) and/or the step v2) and/or the step v3) are carried out under stirring.

Preferably, the step v1) comprises:
v11) providing a hydrolysis product containing the compound of Formula (IV), regulating its pH to 8.5-10 (for example, it can be 8.5, 8.6, 8.7, 8.8, 8.9, 9.0, 9.1, 9.2, 9.3, 9.4, 9.5, 9.6, 9.7, 9.8, 9.9, 10 or any value between any two of these values), and controlling the temperature at 10-15° C. (such as: 10, 11, 12, 13, 14, 15° C. or any value between any two of these values);
v12) adding TEMPO, bicarbonate and bromide, then adding a hypochlorite solution, controlling the temperature at 10-15° C. (such as: 10, 11, 12, 13, 14, 15° C. or any value between any two of these values), and maintaining the pH of the reaction system at 8.5-10 (for example, it can be 8.5, 8.6, 8.7, 8.8, 8.9, 9.0, 9.1, 9.2, 9.3, 9.4, 9.5, 9.6, 9.7, 9.8, 9.9, 10 or any value between any two of these values);
v13) raising the temperature to 20-30° C. (such as: 20, 21, 22, 23, 24, 25, 26, 27, 28, 29, 30° C. or any value between any two of these values), and reacting for 10-16 h (such as: 10, 11, 12, 13, 14, 15, 16 h or any value between any two of these values); and/or
The step v4) comprises:
v41) extracting the reaction product of the step v3) with an extraction agent to obtain an extract;
v42) removing the extraction agent from the extract to obtain a crude product; and
v43) crystalizing the crude product in a crystallization solvent and separating the solid to obtain caronic acid.

Preferably, the hydrolysis product containing the compound of Formula (IV) in the step v11) is an aqueous solution of the compound of Formula (IV), preferably a 28-38 wt % aqueous solution. For example, the concentration of the aqueous solution can be 28, 29, 30, 31, 32, 33, 34, 35, 36, 37, 38 wt % or any value between any two of these values; and/or
The regulation of pH to 8.5-10 in the step v12) is realized by adding sulfuric acid; and/or
The step v12) comprises first reducing the temperature to 10° C. after adding TEMPO, bicarbonate and bromide, and then adding a hypochlorite solution; and/or
The hypochlorite solution in the step v12) is a hypochlorite aqueous solution, preferably an 8-13 wt % hypochlorite aqueous solution, more preferably a 12 wt % hypochlorite aqueous solution. For example, the concentration of the hypochlorite aqueous solution can be 8, 9, 10, 11, 12, 13 wt % or any value between any two of these values; and/or
The maintenance of pH at 8.5-10 in the step v12) is realized by adding a liquid alkali; and/or
The extraction in the step v42) is carried out at 30-45° C. For example, the temperature can be 30, 31, 32, 33, 34, 35, 36, 37, 38, 39, 40, 41, 42, 43, 44, 45° C. or any value between any two of these values; and/or
The crystallization solvent in the step v43) contains:
water; and
alcohol, preferably methanol;
wherein, the weight ratio of water and alcohol is 1:(0.1-0.2).

### Preferred Embodiment

Preferred embodiments of the present invention are further described in detail below. The descriptions below are exemplary and are not limitations of the present invention, and any other similar circumstances fall within the scope of protection of the present invention.

### Raw Materials

Isopentenol purchased from China Catalyst, with designation ZCMHB211107.

Potassium carbonate purchased from Dayang Biotech, with designation 20211228.

Acetic anhydride purchased from Ningbo Wanglong Tech Co., Ltd., with designation 072204111.

Glycine ethyl ester hydrochloride purchased from Jiangsu Youpu Technology Co., Ltd., with designation D20220204019.

Dichloroethane purchased from Shanghai Chlor-Alkali Chemical Co., Ltd., with designation 20211204.

Sodium nitrite purchased from Shijiazhuang Fengshan Chemical Co., Ltd., with designation 12202181.

Catalyst; and A 1:1 complex of cuprous trifluoromethanesulfonate and benzonitrile, purchased from ABA Chemicals Corporation, with designation 2202010.

Sodium hydroxide purchased from Juhua Group Corporation, with designation 20220039.

Sulfuric acid purchased from Anhui Huaertai Chemical Co., Ltd., with designation LS2202192.

TEMPO purchased from Nanjing Hike Biological Technology Co., Ltd., with designation LISKON20220108.

Sodium bicarbonate purchased from Henan Zhongyuan Chemical Co., Ltd., with designation 20211203.

Potassium bromide purchased from Shandong Tansins Chemical Co., Ltd., with designation 20211206.

Sodium hypochlorite purchased from Leping Yongli Chemical Co., Ltd., with designation 2022030311.

Sodium sulfite purchased from Jiangsu Yazhong Chemical Co., Ltd., with designation 20211015.

Methanol purchased from SABIC, with designation 202111015.

Sodium acetate purchased from Weifang Qianxian Moke Chemical Co., Ltd., with designation 012201004.

### Reaction Path

Caronic acid and caronic anhydride are prepared in the following paths:

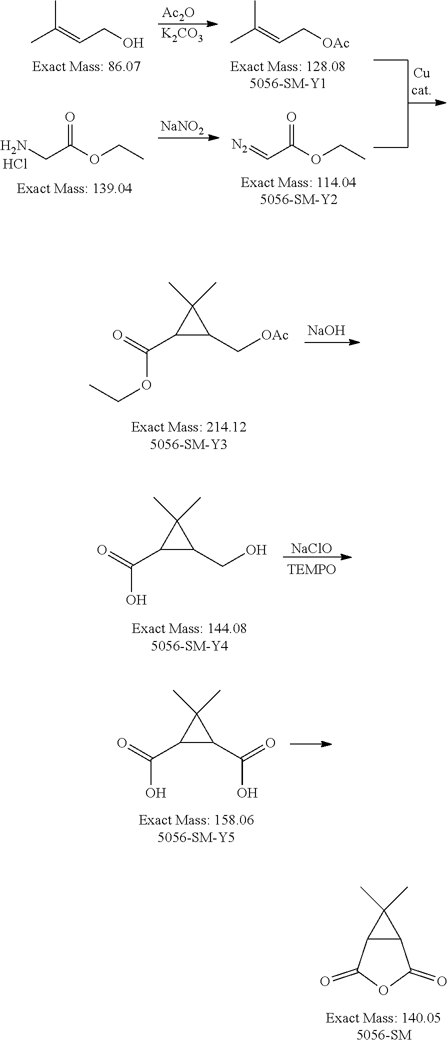

### Embodiment 1 (Optimum Embodiment)

I. Preparation of 5056-SM-Y1 (a product containing a compound of Formula (I′))
1. Add 650 Kg of isopentenol and 57.2 kg of potassium carbonate, stir and raise the temperature to 48° C.;
2. Pump 924 Kg of acetic anhydride into an elevated tank, dropwise add it into a reaction system at this temperature, control the temperature at 48° C. and complete the addition in about 4 h;
3. Stir at 65° C. for 2 h after the addition, take a sample and send it for testing (isopentenol <0.5%);
4. Cool the reaction system to 28° C. and slowly dropwise add 300 kg of water into the reaction system within 1 h;
5. Add 600 kg of water into the system at one time;
6. Continue to stir at 28° C. for 30 min;
7. Let stand, separate an organic phase, obtain an organic phase product and weigh it at 910 Kg (the content of the compound of Formula (I′) is about 100 wt %).

II. Preparation of 5056-SM-Y2 (a product containing a compound of Formula (II′))
1. Dissolve glycine ethyl ester hydrochloride (800 Kg) in 1700 Kg of water;
2. Add 1200 Kg of dichloroethane and 11.4 Kg of acetic acid;
3. Reduce the internal temperature to 8° C.;
4. Dropwise add a solution prepared with 480 Kg of sodium nitrite and 800 Kg of water and pump it to a high level;
5. Dropwise add a sodium nitrite aqueous solution, control the internal temperature at 10° C. and complete the addition in about 4 h;
6. Stir at 10° C. for 1 h after the addition;
7. Stratify, transfer the oil layer out of the reactor and maintain the temperature at 5° C.; extract the water phase with 400 Kg of dichloroethane once, merge the organic phases, and obtain an organic phase product at a weight of 2254 Kg (including 654 Kg of the compound of Formula (II′)).

III. Preparation of 5056-SM-Y3 (a compound of Formula (III′))
1. Add the organic phase product (889 Kg) of the above step I and a catalyst (7.2 Kg, a 1:1 complex of cuprous trifluoromethanesulfonate and benzonitrile) to a reactor. Raise the temperature to 90° C.;
2. Dropwise add 2254 Kg of the organic phase products of the above step II (composed of 654 Kg of 5056-Y2 and 1600 Kg of dichloroethane) to the system within about 8-10 h. During the reaction, a large amount of nitrogen is released;
3. Continue to stir for 30 min after the addition;
4. Distill under vacuum by rotating the reactor, recover dichloroethane, and collect a fraction of 117-120° C./1 kPa to obtain about 737 Kg of a compound of Formula (III′).

IV. Preparation of 5056-SM-Y4 (hydrolysis product containing the compound of Formula (IV))
1. 920 Kg of 5056-Y3 (i.e., the compound of Formula (III′)) prepared in the above step III, 2208 Kg of 50% ethanol, and 1363 Kg of 30% sodium hydroxide aqueous solution;
2. Stir and raise the temperature to 65° C., maintain the temperature for 2 h, take a sample and test it;
3. Distill under vacuum to get ethanol in a volume of about 2,200 L, detect the residual amount of ethanol, take 5 mL of the reaction system sample, add 20 mL of ethyl acetate for extraction, and detect the residual amount of ethanol; if the ethanol in the sample is less than 0.2%, then proceed to the next step; if ethanol ≥0.2%, then continue to add water to take away ethanol;
4. Convert all into a hydrolysis product and obtain a hydrolysis product containing a compound of Formula (IV), which is about 2040 Kg of aqueous solution (including 619 Kg of the compound of Formula (IV), 352 Kg of sodium acetate and 1069 Kg of water).

V. Preparation of 5056-SM-Y5 (caronic acid)
1. Cool the hydrolysis product (1020 Kg) containing the compound of Formula (IV) obtained in the above step IV to 15° C., and add 50% sulfuric acid (about 50 Kg) to regulate PH to 9 at 10° C.;
2. Add 9.21 Kg of TEMPO, 192 Kg of sodium bicarbonate and 16.10 Kg of potassium bromide, stir for 1 h and cool to 10° C.;
3. Start to dropwise add about 2800 Kg of a 12% sodium hypochlorite aqueous solution, control the temperature at 10° C., and use a liquid alkali to maintain the pH value of the system at 9;
4. After the addition, raise the temperature to 20° C., stir for 1 h, send a sample and test it, and extend the reaction time if the raw material is completely reacted (sum of raw material and transition state ≤3.0%);
5. Reduce the temperature to 10° C., and add 115 Kg of sodium sulfite for regulation until the KI starch test paper does not show color;
6. Add 50% sulfuric acid (about 30 g) to regulate the pH to 1;
7. Raise the temperature to 40° C., extract it with ethyl acetate twice, 700 kg of ethyl acetate each time, and merge the organic phases, extract the water phase with 700 kg of ethyl acetate once again and use the organic phase as the next batch of extraction liquid;
8. Concentrate it to dryness to obtain crude caronic acid;
9. Add 500 Kg of water and 50 Kg of methanol to the crude caronic acid, dissolve them at 60° C., cool to 0° C. for crystallization, centrifuge and dry to obtain 258 Kg of solid caronic acid.

VI. Preparation of 5056-SM (caronic anhydride)
1. Add 1000 Kg of caronic acid, 30 Kg of sodium acetate and 2000 Kg of acetic anhydride to a reactor;
2. Raise the temperature to 135-138° C. and perform reflux for 3 h;
3. After the end of reflux, distill acetic acid under atmospheric pressure, and continue the distillation for 5-6 h after the temperature in the reactor is raised to 140-145° C.;
4. Reduce the temperature to 70-80° C., transfer it to a high-temperature reactor, raise temperature, and collect a fraction with internal temperature less than 165° C. and a fraction of 165-200° C., wherein the fraction of less than 165° C. mainly includes a mixture of acetic anhydride/acetic acid, and the fraction of 165-200° C. mainly includes products and acetic anhydride;
5. Maintain the temperature of the fraction of 165-200° C. at 190-200° C. and react for 4 h;
6. Reduce the temperature to 140-145° C., gradually start the three-stage Roots pump, slowly raise the temperature to 180-185° C. and distill crude caronic anhydride to the crystallization reactor;
7. After the end of distillation, add 500 Kg of isopropyl ether to the crystallization reactor, raise the temperature to 55-60° C., dissolve it, maintain the temperature and stir for 30 min;
8. Reduce the temperature to 5-8° C., maintain the temperature and stir for 5 h;
9. Filter under pressure, transfer the mother liquor to an isopropyl ether recovery kettle, and obtain about 600 Kg of caronic anhydride when no liquid flows out obviously and the two cones are dry.

### Comparison Example 1 (Screening of the Conditions for Preparation of 5056-SM-Y2)

The steps are basically the same as those in Embodiment 1 except that the selection of the catalyst in the steps of preparation of 5056-SM-Y2 is optimized and screened. The result is as shown in Table 1 below:

TABLE 1

| | | | Purity of | Yield of |
|---|---|---|---|---|
| | | Amount of | target | target |
| Batch | Catalyst | catalyst (Kg) | product | product |
| 1 | Sulfuric | 48.45 | 74.3% | 72.33% |
| | acid | | | |
| 2 | Formic acid | 48.45 | 86.7% | 91.17% |
| 3 | Formic acid | 28.5 | 90.2% | 95.1% |
| 4 | Formic acid | 11.4 | 96.58% | 95% |
| 6 | Acetic acid | 11.4 (same as that | 98.6% | About 100% |
| | | in Embodiment 1) | | |
| 7 | No catalyst | 0 | 97.19% | 33% |

As can be seen from Table 1 above, the reaction process of preparing 5056-SM-Y2 has considerable selectivity to catalysts. Although the target product can be prepared by using any of sulfuric acid, formic acid and acetic acid, the catalytic effects of formic acid and acetic acid are obviously better than that of sulfuric acid, and the effect of acetic acid is the best. Further, the content of the catalyst in the reaction system also has a certain influence on the catalytic effect, and the catalytic effect reaches the optimum state in a narrow range of specific proportions.

### Comparison Example 2 (Screening of the Conditions for Preparation of 5056-SM-Y3)

The steps are basically the same as those in Embodiment 1 except that the reaction temperature in the steps of preparation of 5056-SM-Y3 is optimized and screened. The result is as shown in Table 2 below:

TABLE 2

| Batch | Condition | Yield | Remarks |
|---|---|---|---|
| 1 | Reaction temperature 90-95° C. | 60% | Comparison in |
| | (same as that in Embodiment 1) | | reaction |
| 2 | Reaction temperature 60-65° C. | 34% | temperature |

### Comparison Example 3 (Screening of the Conditions for Preparation of 5056-SM-Y5)

The steps are basically the same as those in Embodiment 1 except that the reaction conditions (oxidation conditions and crystallization conditions) in the steps of preparation of 5056-SM-Y5 are optimized and screened. The result is as shown in Table 3 and Table 4 below:

TABLE 3

| | | Residual rate | Yield of |
|---|---|---|---|
| | | of raw | crude |
| Batch | Oxidation conditions | material | product |
| 1 | Sodium hypochlorite 2.0 eq, pH regulated to 8-9, 25-30° C., | 0.6% | 85% |
| | 24 h | | |
| 2 | Sodium hypochlorite 1.0eq + sodium chlorite 0.65eq, pH | 1.5% | 54.3% |
| | regulated to 2-3, 25-30° C., 24 h | | |
| 3 | Sodium hypochlorite 1.0eq + sodium chlorite 0.65eq, pH | 3% | 67.5% |
| | regulated to 8-9, 60-70° C., 24 h | | |
| 4 | 1.1eq sodium hypochlorite, 1.1eq hydrogen peroxide, 25- | 1.5% | 23.4% |
| | 35° C.° C., maintain the temperature overnight | | |
| 5 | 1.1eq sodium hypochlorite, 1.1eq hydrogen peroxide, 0.01eq | 1.2% | 36.4% |
| | ferrous sulfate, 25-35° C.° C., maintain the temperature | | |
| | overnight | | |
| 6 | Dropwise add a 12% sodium hypochlorite aqueous solution | 0.9% | 49.5% |
| | (the chlorine content is 10%, and about 20 g is used). Stir for | | |
| | 20 min. Regulate the pH value to 4-7 and dropwise add 16 g of a | | |
| | 25% sodium chlorite aqueous solution | | |
| 7 | Same as Embodiment 1 | 0.5% | 86% |

As can be seen from Table 3 above, oxidation conditions have a great impact on both raw material residual rate and product yield. The technical scheme of Embodiment 1 can achieve significantly better technical effects in terms of raw material residual rate and crude product yield.

TABLE 4

| Batch | Crystallization solvent | Yield | Purity |
|---|---|---|---|
| 1 | Water | 66.7% | 99.5% |
| 2 | Methanol | 90.4% | 85.6% |
| 3 | Water + methanol (weight ratio 1:20) | 88.6% | 95.2% |
| 4 | Water + methanol (weight ratio 1:10, same as | 85.5% | 99.1% |
| | that in Embodiment 1) | | |
| 5 | Water + methanol (weight ratio 1:5) | 79.3% | 99.4% |
| 6 | Water + methanol (weight ratio 1:4) | 73.4% | 99.3% |
| 7 | Water + ethanol (weight ratio 1:10) | 85.3% | 98.3% |

As can be seen from Table 4 above, the crystallization solvent has a great impact on both product yield and purity. When the technical scheme of Embodiment 1 is used, the best technical effects with ideal yield and purity can be achieved.

### Comparison Example 4 (Screening of the Conditions for Preparation of 5056-SM)

The steps are basically the same as those in Embodiment 1 except that a different recrystallization solvent is used.

TABLE 5

| Batch | Recrystallization solvent | Yield | Purity |
|---|---|---|---|
| 1 | Toluene + petroleum ether (volume ratio 1:3) | 57.4 | 98 |
| 2 | Toluene + petroleum ether (volume ratio 1:1) | 45.6 | 99 |
| 3 | Isopropyl ether (same as Embodiment 1) | 65.3 | 99 |

As can be seen from Table 5 above, the recrystallization solvent has a great impact on both product yield and purity. When isopropyl ether is used, the best technical results with ideal yield and purity can be obtained.

The foregoing examples are illustrative only and intended to explain some of the features of the present disclosure. The attached claims are intended to claim as wide a scope as can be conceived, and the embodiments presented herein are only descriptions of implementation manners selected based on all possible combinations of embodiments. Therefore, the intention of the applicant is that the attached claims are not limited by the selection of examples that illustrate the features of the present application. For example, the term “comprise” and its semantic variants used in the claims also logically include different and varied terms, such as, but not limited to, “essentially composed of” or “composed of”. When required, a number of numerical ranges are provided, and these ranges also include subranges within them. Changes in these ranges are also self-evident to those skilled in the art and shall not be considered to be donated to the public, and such changes shall also be construed, where possible, to be covered by the attached claims. Further, advancement in technology will result in possible equivalents or sub-substitutions not currently considered for reasons of linguistic inaccuracies, and such changes shall also be construed, where possible, to be covered by the attached claims.

1. A method for preparing caronic anhydride, which comprises the following steps:
s1) cyclizing caronic acid to obtain crude caronic anhydride; and
s2) using isopropyl ether to recrystallize the crude caronic anhydride to obtain caronic anhydride;
wherein the step s1) comprises:
s11) causing the reaction mixture prepared by mixing a starting material to react, wherein the starting material contains:
caronic acid
a catalyst; and
a solvent; and
s12) distilling, preferably distilling under atmospheric pressure, the product obtained in the above step s11), to separate and obtain crude caronic anhydride.

2. (canceled)

3. The method according to claim 1, wherein:
in the step s11), the catalyst is one or more of: sodium acetate, sodium hydroxide, sodium bicarbonate and sodium carbonate, preferably sodium acetate; and/or
in the step s11), the solvent is acetic anhydride; and/or
in the step s11), the weight ratio of the caronic acid, the catalyst and the solvent is 1:(0.01-0.05): (1-3), preferably 1:0.03:2; and/or
the step s11) is carried out at 130-140° C., preferably 135-138° C. for 1-5 h, preferably 2-4 h, more preferably 3 h; and/or
the step s12) comprises:
s121) distilling under atmospheric pressure at 140-145° C. the product obtained in the step s11) for 3-8 h to obtain a post-distillation product;
s122) heating and treating the post-distillation product, preferably in a high-temperature reactor, to collect a fraction of 165-200° C.;
s123) maintaining the temperature of the fraction obtained in the step s122) at 190-200° C. for 3-6 h to obtain crude caronic anhydride.

4. The method according to claim 1, wherein the step s2) comprises:
s21) dissolving the crude caronic anhydride in isopropyl ether at a high temperature, preferably 50-70° C., more preferably 55-60° C. to obtain a clear solution; and
s22) cooling the clear solution obtained in the above step s21) to a low temperature, preferably to 3-10° C., more preferably to 5-8° C. to precipitate a solid, i.e., caronic anhydride.

5. The method according to claim 1, wherein the caronic acid is prepared by a method comprising the following steps:
a) causing a compound of Formula (I) to react with a compound of Formula (II) to obtain a compound of Formula (III), 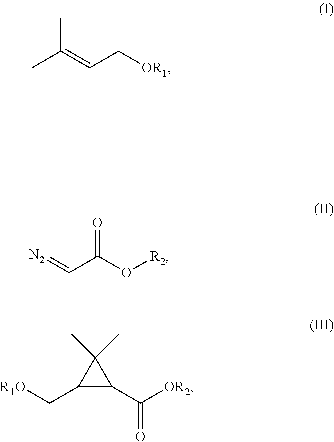
where the R₁ is a protecting group, preferably, the R₁ is selected from an ester protecting group, an alkyl ether protecting group and a silyl ether protecting group, more preferably, the ester protecting group is selected from acetyl, benzoyl and substituted benzoyl, the alkyl ether protecting group is selected from benzyl, triphenyl-methyl and tetrahydropyranyl, and the silyl ether protecting group is selected from trimethylsilyl and dimethyltert-butylsilyl, and most preferably, the protecting group is acetyl;
the R₂ is selected from: alkyl, cycloalkyl, aryl, alkylaryl, heterocyclyl and heteroaryl, preferably, the R₂ is selected from substituted or unsubstituted alkyl, more preferably, the R₂ is selected from unsubstituted alkyl, most preferably, the R₂ is ethyl;
b) hydrolyzing the compound of Formula (III) to obtain a compound of Formula (IV), 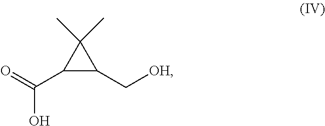
c) using a TEMPO oxidation system to oxidize the compound of Formula (IV) and obtain caronic acid,
the TEMPO oxidation system comprises:
TEMPO;
bicarbonate;
bromide; and
hypochlorite;
wherein
the bicarbonate is sodium bicarbonate; and/or
the bromide is potassium bromide; and/or
the hypochlorite is sodium hypochlorite; and/or
the weight ratio of the compound of Formula (IV), the TEMPO, the bicarbonate, the potassium bromide and the hypochlorite is 1:(0.01-0.05): (0.3-1): (0.01-0.1): (0.9-1.4), preferably 1:(0.02-0.04): (0.5-0.8): (0.02-0.08): (1-1.3), more preferably 1:(0.02-0.04): (0.5-0.7): (0.04-0.06): (1-1.2), most preferably 1:0.03:0.62:0.052:1.08.

6. (canceled)

7. The method according to claim 5, wherein the step a) comprises:
a1) causing the reaction mixture prepared by mixing a starting material to react, wherein the starting material contains:
a compound of Formula (I);
a compound of Formula (II);
a catalyst; and
a solvent; and
a2) separating from the reaction product obtained in the step a1) to obtain the compound of Formula (III);
wherein
in the step a1), the weight ratio of the compound of Formula (I), the compound of Formula (II), the catalyst and the solvent is 1: (0.5-1.0): (0.005-0.015): (1.5-2.5), preferably 1:(0.6-0.8): (0.006-0.01): (1.6-2), more preferably 1: (0.6-0.8): (0.007-0.09): (1.7-1.9), most preferably 1: 0.74:0.008:1.8; and/or
the step a1) is carried out at 85-110° C., preferably 90-95° C.; and/or
the step a1) is carried out under stirring; and/or
the reaction in the step a1) is carried out for 8-16 h; and/or
the catalyst in the step a1) is a copper catalyst, preferably, the copper catalyst is one or more of: metal copper, cuprous chloride, cuprous bromide, cuprous iodide, cuprous trifluoromethanesulfonate, cupric sulfate, cupric acetate, cupric trifluoromethylsulfonyl and cupric chloride, more preferably, the catalyst is cuprous trifluoromethanesulfonate, most preferably, the catalyst is a 1:1 complex of cuprous trifluoromethanesulfonate and benzonitrile; and/or
the solvent is one or more of: dichloroethane, dichloromethane and toluene, preferably, the solvent is dichloroethane.

8. (canceled)

9. The method according to claim 7, wherein:
the step a1) comprises:
a11) in a reactor, adding a compound of Formula (I) and a catalyst; and
a12) adding the solution obtained by dissolving the compound of Formula (II) in the solvent to the reactor and causing it to react; and/or
the step a2) comprises:
a21) distilling under vacuum the reaction product obtained in the step a1) and collecting a fraction to obtain the compound of Formula (111);
wherein
in the step a12), the mass ratio of the compound of Formula (II) and the solvent is 1:(1.5-2.5); and/or
the solvent in the step a12) is dichloroethane; and/or
the addition in the step a12) is carried out in a way of dropwise adding, preferably in a way of completing dropwise adding within 8-16 h; and/or
the distillation under vacuum in the step a21) is carried out by rotating the reactor; and/or
the collection of a fraction is carried out by collecting a fraction of 117-120° C./1 kPa.

10. (canceled)

11. The method according to claim 5, wherein the step b) comprises:
b1) hydrolyzing the reaction mixture prepared by mixing a starting material, wherein the starting material contains:
a compound of Formula (III);
an inorganic base; and
a solvent; and
b2) separating from the reaction product obtained in the step b1) to obtain a compound of Formula (IV);
wherein
in the step b1), the weight ratio of the compound of Formula (III), the inorganic base and the solvent is 1: (0.35-0.5): (2-4), preferably 1:(0.4-0.5): (3-4), more preferably 1:0.44:3.43; and/or
in the step b1), the inorganic base is selected from sodium hydroxide and/or potassium hydroxide, preferably, the inorganic base is sodium hydroxide; and/or
in the step b1), the solvent is selected from water and/or alcohol, preferably, the solvent is a mixture of water and alcohol, more preferably, the solvent is a mixture of water and ethanol and the weight ratio of the water and the ethanol is 1:(1-2); and/or
the step b1) is carried out under stirring; and/or
the step b1) is carried out at 50-65° C.; and/or
the step b1) is carried out for 2-4 h.

12. (canceled)

13. The method according to claim 11, wherein:
the step b1) comprises: mixing the compound of Formula (III), the solvent and the inorganic base solution to set off a hydrolysis reaction; and/or
the step b2) comprises: removing the solvent from the reaction product obtained in the step b1) to obtain a compound of Formula (IV);
wherein
the solvent is a mixture of water and alcohol, preferably, the solvent is a mixture of water and ethanol, more preferably, the solvent is a 30-50 wt % ethanol aqueous solution; and/or
the inorganic base solution is an inorganic base aqueous solution, preferably, the inorganic base solution is a sodium hydroxide aqueous solution, more preferably is a 30-40 wt % sodium hydroxide aqueous solution.

14. (canceled)

15. The method according to claim 5, wherein the step c) comprises:
c1) oxidizing the reaction mixture prepared by mixing a starting material, wherein the starting material contains:
a compound of Formula (IV);
TEMPO;
bicarbonate;
bromide;
hypochlorite; and
a solvent;
c2) adding a sulfite solid and/or a chlorite solid and/or a sulfite solution and/or a chlorite solution to the reaction product of the step c1);
c3) regulating the pH of the reaction system to 1-2; and
c4) separating from the product of the step c3) to obtain caronic acid;
wherein
the solvent in the step c1) is water; and/or
the step c1) is carried out at 20-30° C.; and/or
the step c1) is carried out at pH 8.5-10; and/or
the oxidation reaction in the step c1) is carried out for 10-16 h; and/or
the step c2) is carried out at 10-15° C.; and/or
the sulfite in the step c2) is sodium sulfite; and/or
the sulfite solution in the step c2) is a sodium sulfite aqueous solution; and/or
the chlorite in the step c2) is sodium chlorite; and/or
the chlorite solution in the step c2) is a sodium chlorite aqueous solution; and/or
the step c3) is realized by adding sulfuric acid; and/or
the step c1) and/or the step c2) and/or the step c3) are carried out under stirring.

16.-18. (canceled)

19. The method according to claim 1, wherein the caronic acid is prepared by a method comprising the following steps:
i) causing the reaction mixture prepared by mixing a starting material to react and separating the reaction product to obtain an organic phase product containing a compound of Formula (I′), wherein the starting material contains:
isopentenol;
acetic anhydride; and
carbonate; 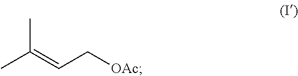
ii) causing the reaction mixture prepared by mixing a starting material to react and separating the reaction product to obtain an organic phase product containing a compound of Formula (II′), wherein the starting material contains:
glycine ethyl ester hydrochloride;
sodium nitrite;
a catalyst; and
a solvent; 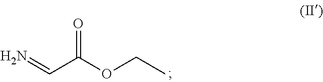
iii) mixing the organic phase product of the step i) and the organic phase product of the step ii), causing them to react and separating the reaction product to obtain a compound of Formula (III′), 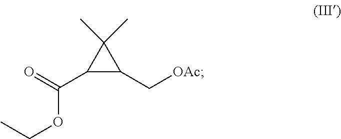
iv) hydrolyzing the compound of Formula (III′) to obtain a hydrolysis product containing the compound of Formula (IV), 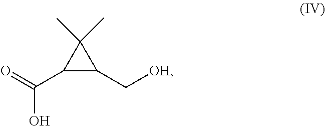
v) using a TEMPO oxidation system to oxidize the hydrolysis product of the step iv) and separating the reaction product to obtain caronic acid.
Preferably, the TEMPO oxidation system comprises:
TEMPO;
bicarbonate;
bromide; and
hypochlorite.

20. The method according to claim 19, wherein:
in the step i), the weight ratio of isopentenol, acetic anhydride and carbonate is 1:(1.25-1.65): (0.09-0.15), preferably 1:1.42:0.09; and/or
the carbonate in the step i) is potassium carbonate; and/or
the reaction in the step i) is carried out at 65° C.-75° C.; and/or
the reaction in the step i) is carried out for 5-8 h, preferably 6-7 h; and/or
in the organic phase product of the step i), the content of acetic acid is less than 0.6%, preferably less than 0.1%, preferably is realized by means of alkaline washing; and/or
in the step ii), the weight ratio of glycine ethyl ester hydrochloride, sodium nitrite, the catalyst and the solvent is 1:(0.5-0.65): (0.012-0.025): (3.1-5.0), preferably 1:0.6:0.014:4.6; and/or
the catalyst in the step ii) is formic acid and/or acetic acid, preferably acetic acid; and/or
the solvent in the step ii) is water and/or dichloroethane, preferably, the solvent is a mixture of water and dichloroethane, most preferably, the solvent is a mixture of water and dichloroethane and the weight ratio of the water and the dichloroethane is 1:(0.3-0.8), preferably 1:0.48; and/or
the reaction in the step ii) is carried out at 5-15° C.; and/or
the reaction in the step iii) is carried out for 8-16 h; and/or
in the step iii), the weight ratio of the organic phase product of the step i) and the organic phase product of the step ii) is 1:(2-3), preferably 1:2.5; and/or
the bicarbonate in the step v) is sodium bicarbonate; and/or
the bromide in the step v) is potassium bromide; and/or
the hypochlorite in the step v) is sodium hypochlorite; and/or
The weight ratio of the hydrolysis product of the step iv), the TEMPO, the bicarbonate, the potassium bromide and the hypochlorite in the step v) is 1:(0.01-0.05): (0.1-0.3): (0.01-0.03): (0.1-0.5), preferably 1:(0.008-0.01): (0.1-0.3): (0.01-0.03): (0.2-0.4), more preferably 1:0.009:0.19:0.016:0.33.

21. The method according to claim 19, wherein:
the step i) comprises:
i1) mixing isopentenol with carbonate;
i2) adding acetic anhydride to the mixture of the step i1) and causing them to react;
i3) adding water to the reactants of the step i2) and separating the organic phase to obtain the organic phase product;
wherein
the step i1) is carried out under stirring; and/or
the step i1) is carried out at 45-55° C., preferably 48-52° C.; and/or
the step i2) comprises dropwise adding acetic anhydride at 48-75° C. within 4-8 h and then stirring at 65-75° C. for 2-4 h; and/or
the step i3) comprises:
i31) cooling the reaction system to 20-35° C. preferably 28-32° C. dropwise adding water to the reaction system within 1-3 h, then adding water to the system at one time and continuing to stir at 20-35° C. preferably 28-32° C. for 0.5-2 h, preferably 30 min;
i32) letting stand and separating an organic phase to obtain the organic phase product.

22. (canceled)

23. The method according to claim 19, wherein the step ii) comprises:
ii1) dissolving glycine ethyl ester hydrochloride in a solvent;
ii2) adding a solvent and a catalyst to the mixture obtained in the step ii1);
ii3) adding a sodium nitrite solution to the mixture obtained in the step ii2) and causing them to react; and
ii4) separating from the product obtained in the step ii3) to obtain the organic phase product;
wherein
in the step ii1), the weight ratio of the glycine ethyl ester hydrochloride and the solvent is 1:(1-2.4), preferably 1:2.1; and/or
the solvent in the step ii1) is water; and/or
in the step ii2), the weight ratio of the solvent and the catalyst is 1:(0.0085-0.015), preferably 1:0.0095; and/or
the solvent in the step ii2) is dichloroethane; and/or
the sodium nitrite solution in the step ii3) is a sodium nitrite aqueous solution, preferably a 20-50 wt % sodium nitrite aqueous solution, more preferably a 30-40 wt % sodium nitrite aqueous solution; and/or
the step ii3) comprises: dropwise adding a sodium nitrite aqueous solution, controlling the temperature at 5-15° C. completing the addition in about 4-8 h and then stirring again at 5-15° C. for 1-3 h; and/or
the step ii4) comprises: splitting the product obtained in the step ii3) into different phases, preferably using dichloroethane to extract the water phase and merging the organic phases to obtain the organic phase product.

24. (canceled)

25. The method according to claim 19, wherein the step iii) comprises:
iii1) causing the reaction mixture prepared by mixing a starting material to react, wherein the starting material contains:
the organic phase product of the step i);
the organic phase product of the step ii); and
a catalyst; and
iii2) separating from the reaction product obtained in the step iii1) to obtain the compound of Formula (III′);
wherein
in the step iii1), the weight ratio of the organic phase product of the step i), the organic phase product of the step ii) and the catalyst is 1:(2-3): (0.005-0.015), preferably 1:2.53:0.0081; and/or
the step iii1) is carried out at 85-110° C., preferably 90-95° C.; and/or
the step iii1) is carried out under stirring; and/or
the reaction in the step iii1) is carried out for 8-16 h; and/or
the catalyst in the step iii1) is a copper catalyst, preferably, the copper catalyst is one or more of: metal copper, cuprous chloride, cuprous bromide, cuprous iodide, cuprous trifluoromethanesulfonate, cupric sulfate, cupric acetate, cupric trifluoromethylsulfonyl and cupric chloride, more preferably, the catalyst is cuprous trifluoromethanesulfonate, most preferably, the catalyst is a 1:1 complex of cuprous trifluoromethanesulfonate and benzonitrile.

26. (canceled)

27. The method according to claim 25, wherein:
the step iii1) comprises:
iii11) in a reactor, adding the organic phase product of the step i) and a catalyst; and
iii12) adding the organic phase product of the step ii) to the reactor and causing them to react; and/or
the step iii2) comprises:
iii21) distilling under vacuum the reaction product obtained in the step iii1) and collecting a fraction to obtain the compound of Formula (III′);
wherein
in the organic phase product of the step i) in the step iii11), the concentration of the compound of Formula (I′) is 90-100 wt %; and/or
in the organic phase product of the step ii) in the step iii12), the concentration of the compound of Formula (II′) is 20-40 wt %, preferably 29 wt %; and/or
the addition in the step iii12) is carried out in a way of dropwise adding, preferably in a way of completing dropwise adding within 8-16 h; and/or
the distillation under vacuum in the step iii21) is carried out by rotating the reactor; and/or
the collection of a fraction in the step iii21) is carried out by collecting a fraction of 117-120° C./1 kPa.

28. (canceled)

29. The method according to claim 19, wherein the step iv) comprises:
iv1) hydrolyzing the reaction mixture prepared by mixing a starting material, wherein the starting material contains:
a compound of Formula (III′);
an inorganic base; and
a solvent; and
iv2) obtaining a hydrolysis product containing the compound of Formula (IV) from the reaction product obtained in the step iv1);
wherein
in the step iv1), the weight ratio of the compound of Formula (III′), the inorganic base and the solvent is 1: (0.35-0.5): (2-4), preferably 1:(0.4-0.5): (3-4), more preferably 1:0.44:3.43; and/or
in the step iv1), the inorganic base is selected from sodium hydroxide and/or potassium hydroxide, preferably, the inorganic base is sodium hydroxide; and/or
in the step iv1), the solvent is selected from water and/or alcohol, preferably, the solvent is a mixture of water and alcohol, more preferably, the solvent is a mixture of water and ethanol and the weight ratio of the water and the ethanol is 1:(1-2); and/or
the step iv1) is carried out under stirring; and/or
the step iv1) is carried out at 50-65° C.; and/or
the step iv1) is carried out for 2-4 h.

30. (canceled)

31. The method according to claim 29, wherein:
the step iv1) comprises: mixing the compound of Formula (III′), the solvent and the inorganic base solution to set off a hydrolysis reaction; and/or
the step iv2) comprises: removing alcohol from the reaction product obtained in the step b1) to obtain an aqueous solution of the compound of Formula (IV), i.e., a hydrolysis product containing the compound of Formula (IV);
wherein
the solvent is a mixture of water and alcohol, preferably, the solvent is a mixture of water and ethanol, more preferably, the solvent is a 30-50 wt % ethanol aqueous solution; and/or
the inorganic base solution is an inorganic base aqueous solution, preferably, the inorganic base solution is a sodium hydroxide aqueous solution, more preferably is a 30-40 wt % sodium hydroxide aqueous solution; and/or
the content of the alcohol in the hydrolysis product containing the compound of Formula (IV) is less than 0.5 wt %, preferably less than 0.2 wt %.

32. (canceled)

33. The method according to claim 19, wherein the step v) comprises:
v1) oxidizing the reaction mixture prepared by mixing a starting material, wherein the starting material contains:
a hydrolysis product containing the compound of Formula (IV);
TEMPO;
bicarbonate;
bromide; and
hypochlorite;
v2) adding a sulfite solid and/or a chlorite solid and/or a sulfite solution and/or a chlorite solution to the reaction product of the step v1);
v3) regulating the pH of the reaction system to 1-2; and
v4) separating from the product of the step v3) to obtain caronic acid;
wherein
the step v1) is carried out at 20-30° C.; and/or
the step v1) is carried out at pH 8.5-10; and/or
the oxidation reaction in the step v1) is carried out for 10-16 h; and/or
the step v2) is carried out at 10-15° C.; and/or
the sulfite in the step v2) is sodium sulfite; and/or
the sulfite solution in the step v2) is a sodium sulfite aqueous solution; and/or
the chlorite in the step v2) is sodium chlorite; and/or
the chlorite solution in the step v2) is a sodium chlorite aqueous solution; and/or
the step v3) is realized by adding sulfuric acid; and/or
the step v1) and/or the step v2) and/or the step v3) are carried out under stirring.

34. (canceled)

35. The method according to claim 33, wherein:
the step v1) comprises:
v11) providing a hydrolysis product containing the compound of Formula (IV), regulating its pH to 8.5-10 and controlling its temperature at 10-15° C.;
v12) adding TEMPO, bicarbonate and bromide, then adding a hypochlorite solution, controlling the temperature at 10-15° C., and maintaining the pH of the reaction system at 8.5-10;
v13) raising the temperature to 20-30° C. and reacting for 10-16 h; and/or
the step v4) comprises:
v41) extracting the reaction product of the step v3) with an extraction agent to obtain an extract;
v42) removing the extraction agent from the extract to obtain a crude product; and
v43) crystalizing the crude product in a crystallization solvent and separating the solid to obtain caronic acid.

36. The method according to claim 35, wherein:
the hydrolysis product containing the compound of Formula (IV) in the step v11) is an aqueous solution of the compound of Formula (IV), preferably a 28-38 wt % aqueous solution; and/or
the regulation of pH to 8.5-10 in the step v12) is realized by adding sulfuric acid; and/or
the step v12) comprises first reducing the temperature to 10° C. after adding TEMPO, bicarbonate and bromide, and then adding a hypochlorite solution; and/or
the hypochlorite solution in the step v12) is a hypochlorite aqueous solution, preferably an 8-13 wt % hypochlorite aqueous solution, more preferably a 12 wt % hypochlorite aqueous solution; and/or
the maintenance of pH at 8.5-10 in the step v12) is realized by adding a liquid alkali; and/or
the extraction in the step v42) is carried out at 30-45° C.; and/or
the crystallization solvent in the step v43) contains:
water; and
alcohol, preferably methanol;
wherein, the weight ratio of water and alcohol is 1:(0.1-0.2).
